(19) United States
(12) Patent Application Publication
(10) Pub. No.: US 2014/0350355 A1
(43) Pub. Date: Nov. 27, 2014

(54) MONITORING AND MANAGING SLEEP BREATHING DISORDERS

(71) Applicant: P-Tech HM Ltd., Ramat-Yishai (IL)

(72) Inventors: Ilan AISIC, Kiryat-Tivon (IL); Naveh TOV, Haifa (IL)

(73) Assignee: P-Tech HM Ltd., Ramat-Yishai (IL)

(21) Appl. No.: 14/287,270

(22) Filed: May 27, 2014

Related U.S. Application Data
(60) Provisional application No. 61/827,686, filed on May 27, 2013.

(51) Int. Cl. A61B 5/00; A61B 5/145; A61B 5/113; A61B 5/0205; A61B 7/00; A61B 7/04

(52) U.S. Cl. CPC A61B 5/0022; A61B 7/003; A61B 5/4818; A61B 5/7282; A61B 7/04; A61B 5/1135; A61B 5/0205; A61B 5/14542; A61B 5/746; A61B 5/7203; A61B 5/0004; A61B 5/024
USPC 600/301; 600/586; 600/484

(57) ABSTRACT

A method of monitoring a patient to identify a sleep breathing disorder, comprising:
a) Collecting, using a handheld monitoring device, a plurality of respiratory sounds of a patient using one or more sensors which are connected to the handheld monitoring device.
b) Recording environment noise during the sleep session and averaging the environment noise to set an average noise level.
c) Calculating a dynamic threshold according to the average noise level.
d) Deriving a plurality of amplitudes from the plurality of respiratory sounds according to the dynamic threshold.
e) Identifying a sleep breathing disorder event by analyzing a pattern of the plurality of amplitudes.

### RELATED APPLICATION

This application claims the benefit of priority under 35 USC 119(e) of U.S. Provisional Patent Application No. 61/827,686 filed May 27, 2013, the contents of which are incorporated herein by reference in their entirety.

### BACKGROUND

The present invention, in some embodiments thereof, relates to monitoring and managing sleep breathing disorder events of patients and, more specifically, but not exclusively, to monitoring the patients using handheld client terminals and prioritizing the patients for diagnosis by one or more caregivers.

Sleep breathing disorders, for example, sleep apnea and/or sleep hypopnea, during sleep are common health problems experienced by large parts of the human population. Sleep breathing disorder is a state in which the breathing cycle is halted for extended periods of time. Sleep breathing disorder may be caused by a blockage in the air passageways which may prevent air to flow freely in and/or out of the lungs. Sleep breathing disorder is usually accompanied with changes in vital signs, for example, excessive respiratory sounds, increased heart beat rate, decreased oxygenation level in the blood stream and/or excessive movement while sleeping. Excessive respiratory sound may include, for example, snoring, wheezing, rapid breathing and/or biphasic breathing. Sleep breathing disorder affects the sleep quality and/or sleep duration and may result in insufficient rest for the body and is associated with multiple comorbidities, for example, hypertension, cardiovascular diseases, diabetes, obesity, ADHD and/or depression. In addition people experiencing Sleep breathing disorder effects may be involved in road and/or work accidents at rates of up to 10 times more than average.

Monitoring sleep breathing disorders for the purpose of diagnosis and treatment may include collecting the plurality of vital signs while the patient is sleeping. Traditionally, diagnosing patients suffering from sleep breathing disorders is done at sleep laboratories where the patient goes through a sleep study (also may be known as Polysomnogram) during which he is monitored and records of Sleep breathing disorders are collected and stored. Monitoring may include a plurality of measured vital signs, for example, respiratory sound, chest movement, heart beat rate, blood oxygenation level and/or patient movement. The records are analyzed and a diagnosis is generated for a treatment. This approach presents several difficulties such as, short sessions, unnatural sleeping environment and/or high costs. As the sleep session is limited to one session at worst case or very few sessions at best case, limited sleep data is collected for analysis which may result in inaccurate diagnosis. In addition, costs of the sleep sessions at the sleep laboratories may be high, making the service inaccessible to large parts of the population.

Home monitors are available which enable monitoring multiple sleep sessions of the patients at his natural sleep environment. These home monitors provide higher volumes of sleep breathing disorder information to allow for better and/or more accurate diagnosis of the patient. However, use of these home monitors may be limited to few sleep sessions due to their cost, which may be quite substantial, making their use susceptible to inaccuracies and incomplete which may be overcome using longer analysis periods. The cost may further make these home monitors inaccessible to many patients.

All together, the majority of population suffering of Sleep breathing disorders has never been diagnosed. Moreover, there is usually no follow up monitoring of Sleep breathing disorders for patients who are treated, for example, through oral appliances and/or non-automated continuous positive air pressure (CPAP) devices and/or medications and/or have undergone a medical treatment and/or surgery. Follow up information may be available from the patients themselves who may be reporting of their condition and/or subjective impressions. The lack of objective and/or accurate follow up Sleep breathing disorders monitoring information presents a problem to the caregivers, for example, doctors, clinicians, nurses and/or other medical personnel with respect to assessing the compliance and/or efficacy of the treatment.

Managing large quantities of Sleep breathing disorders information collected from multiple patients using home monitors may present a problem for the caregivers, for example, doctors, clinicians, nurses and/or other medical personnel who need to analyze the collected Sleep breathing disorders information and provide diagnosis and/or treatment.

### SUMMARY

According to some embodiments of the present invention there are provided methods of monitoring a patient to identify a sleep breathing disorder. Managing the suspected sleep breathing disorder events starts with collecting, using a handheld monitoring device, a plurality of respiratory sounds of a patient during a sleep session using one or more sensors which are connected to the handheld monitoring device. The environment noise is recorded during the sleep session and averaged to set an average noise level. A dynamic threshold is calculated according to the average noise level. A plurality of amplitudes is derived from the plurality of respiratory sounds according to the dynamic threshold to identify one or more sleep breathing disorder events by analyzing a pattern of the plurality of amplitudes.

Optionally, identifying the one or more sleep breathing disorder event includes avoiding a false detection of the one or more sleep breathing disorder events by analyzing the pattern.

Optionally, the pattern is transmitted to one or more caregivers for diagnosis of the one or more sleep breathing disorder events by analyzing the pattern.

Optionally, one or more alerts are generated to the one or more caregivers when the one or more sleep breathing disorder event occurs.

Optionally, stimulation is initiated to the patient to cause the patient to exit the one or more sleep breathing disorder events by exciting the patient through a physical intervention.

Optionally, the one or more sensors include a microphone sampling the respiratory sound.

Optionally, the plurality of amplitudes is corrected to reduce fading effects of the microphone by analyzing timing characteristics of previous amplitudes of the plurality of amplitudes.

Optionally, the plurality of respiratory sounds include one or more member of a group consisting of: inhalation sound, exhalation sound, respiratory pause, snore sound and wheeze sound.

Optionally, a plurality of chest movement measurements is collected to identify excessive chest movement which is indicative of the one or more sleep breathing disorder event by analyzing the plurality of chest movement measurements.

Optionally, a plurality of heart beat rate measurements is collected to identify increased heart beat rate which is indicative of the one or more sleep breathing disorder event by analyzing the plurality of heart beat rate measurements.

Optionally, a plurality of blood oxygenation level measurements is collected to identify decreased blood oxygenation level which is indicative of the one or more sleep breathing disorder event by analyzing the plurality of blood oxygenation level measurements.

Optionally, the handheld monitoring device executes at one or more software application programs for collecting the plurality of respiratory sounds, recording the environment noise, calculating the dynamic threshold, deriving the plurality of amplitudes, identifying the one or more sleep breathing disorders and transmitting the pattern to one or more caregivers.

According to some embodiments of the present invention, there are provided methods of prioritizing diagnosis of sleep breathing disorder events of a plurality of patients. A plurality of sleep session records is collected from a plurality of patients using a plurality of client terminals. A plurality of sleep breathing disorder events are identified by analyzing the plurality of sleep session records. The sleep breathing disorder events are prioritized in a priority order according to severity of the plurality of sleep breathing disorder events. One or more caregivers are notified of one or more of the plurality of sleep breathing disorder events in the priority order by transmitting one or more alerts to the one or more caregivers.

Optionally, the one or more caregivers diagnose the plurality of sleep breathing disorder by analyzing the plurality of sleep session records.

Optionally, the one or more caregivers adjust a plurality of alert rules to define at least one condition for transmitting the one or more alerts.

Optionally, the plurality of alert rules is set automatically to default values.

Optionally, the priority order is set automatically according to sleep breathing disorder history of the plurality of patients.

According to some embodiments of the present invention, there are provided systems for monitoring sleep breathing disorders of patients. The system includes a monitoring module, a processing module, an analysis module, a management module and an alert module. The monitoring module which collects a plurality of respiratory sounds of one or more patients using one or more sensors. The processing module which identifies a plurality of amplitudes derived from the plurality of respiratory sounds by dynamically adjusting a threshold to reduce environment noise. The analysis module which identifies one or more sleep breathing disorder events by analyzing a pattern of the plurality of amplitudes. The management module which aggregates the pattern for presentation to one or more caregivers for diagnosis. The alert module which informs the one or more caregivers of the one or more sleep breathing disorders by generating one or more alerts to the one or more caregivers.

Optionally, the management module includes a user interface module for presenting the presentation to said at least one caregiver.

Optionally, the one or more caregivers adjust one or more alert conditions by adjusting a plurality of alert rules through the management module.

Optionally, the management module automatically sets the plurality of alert rules according to sleep breathing disorder history of the one or more patients. The sleep breathing disorder history is stored by the management module.

Optionally, the system includes a stimulation module which initiates stimulation to the one or more patients to cause the one or more patients to exit the one or more sleep breathing disorder events. The stimulation is generated through one or more stimulation devices.

Optionally, the system is a distributed system comprising of one or more handheld devices which identify the one or more sleep breathing disorder events by monitoring the one or more patients and one or more client terminals used by one or more caregivers for managing the one or more sleep breathing disorder events. The one or more handheld devices and the one or more client terminals communicate with each other using one or more of a plurality of networks.

Optionally, the distributed system includes a central unit which provides management services. The central unit communicates with the one or more handheld devices and the one or more client terminals using one or more of the plurality of networks.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an exemplary system for monitoring and managing sleep breathing disorders, according to some embodiments of the present invention;

FIG. 2 is a flowchart of an exemplary process of monitoring and managing sleep breathing disorder events in which processing and analysis is performed at the monitoring device, according to some embodiments of the present invention;

FIG. 2A is a flowchart of an exemplary process of monitoring and managing sleep breathing disorder events in which processing and analysis is performed at the caregiver client terminal, according to some embodiments of the present invention;

FIG. 2B is a flowchart of an exemplary process of monitoring and managing sleep breathing disorder events in which processing and analysis is performed at the central unit, according to some embodiments of the present invention;

FIG. 3 a schematic illustration of a typical breathing cycle and an exemplary excessive respiratory sound level cycle;

FIG. 4 is a schematic illustration of an exemplary respiratory cycle construction, according to some embodiments of the present invention;

FIG. 5A is a schematic illustration of an exemplary computation for reducing microphone fading effect, according to some embodiment of the present invention;

FIG. 5B is a schematic illustration of an exemplary computation for reducing microphone fading effect for a processed respiratory cycle, according to some embodiment of the present invention;

FIG. 6A is a schematic illustration of identifying an apnea event during an exemplary respiratory cycle containing snoring and/or wheezing sounds, according to some embodiment of the present invention;

FIG. 6B is a schematic illustration of identifying a hypopnea event during an exemplary respiratory cycle containing snoring and/or wheezing sounds, according to some embodiment of the present invention;

FIG. 7 is a schematic illustration of identifying a suspected apnea event using multiple sensors, according to some embodiment of the present invention;

FIG. 8 is a schematic illustration of identifying a suspected hypopnea event using multiple sensors, according to some embodiment of the present invention;

FIG. 9 is a schematic illustration of exemplary modules for implementing an exemplary system for monitoring and managing sleep breathing disorders, according to some embodiment of the present invention;

FIG. 10 which is a screen capture of a monitored exemplary sleep session as presented by an exemplary user interface of an exemplary monitoring device, according to some embodiment of the present invention;

FIG. 11 is a screen capture of a result of an exemplary stimulation during a sleep session as presented by an exemplary user interface of an exemplary monitoring device, according to some embodiment of the present invention;

FIG. 12 is a screen capture of an exemplary candidate for an apnea event as presented by an exemplary user interface of an exemplary monitoring device, according to some embodiment of the present invention;

FIG. 13 is a screen capture of an exemplary candidate for a hypopnea event as presented by an exemplary user interface of an exemplary monitoring device, according to some embodiment of the present invention;

FIG. 14 is a screen capture of a monitored exemplary sleep session with multiple sensors as presented by an exemplary user interface of an exemplary monitoring device, according to some embodiment of the present invention;

FIG. 15 is a screen capture of a monitored exemplary sleep session with multiple sensors as presented by an exemplary user interface of an exemplary client terminal used by a caregiver, according to some embodiment of the present invention;

FIG. 16 is a screen capture of an exemplary statistics summary of a monitored exemplary sleep session consisting as presented by an exemplary user interface of an exemplary monitoring device, according to some embodiment of the present invention;

FIG. 17 is a screen capture of a zoom in view of a monitored exemplary sleep session as presented by an exemplary user interface of an exemplary monitoring device, according to some embodiment of the present invention; and

FIG. 18 is a screen capture of a suspected apnea event identified during a monitored exemplary sleep session using multiple sensors as presented by an exemplary user interface of an exemplary monitoring device, according to some embodiment of the present invention.

### DETAILED DESCRIPTION

The present invention, in some embodiments thereof, relates to monitoring and managing sleep breathing disorder events of patients and, more specifically, but not exclusively, to monitoring the patients using handheld client terminals and prioritizing the patients for diagnosis by one or more caregivers.

According to some embodiments of the present invention, there are provided systems and methods for dynamically monitoring sleep breathing disorder events of a plurality of patients. The sleep breathing disorder events are identified by a handheld monitoring device which analyzes patterns of respiratory sounds that are collected from one or more sensors monitoring the patient. The respiratory sounds may be processed in order to create a coherent pattern by improving signal quality and/or reduce noise effects. Processing the respiratory sounds may include smoothing the waveform of the respiratory sounds by averaging and/or interpolating the respiratory sounds, reducing environment noise by adjusting a dynamic threshold level and/or reducing microphone fading effects. Additional monitoring information, for example: chest movement, heart beat rate and/or blood oxygenation may be collected and analyzed to support identification of the sleep breathing disorder events.

According to some embodiments of the present invention, there are provided systems and methods for managing and/or prioritizing sleep sessions' information collected from a plurality of monitoring devices, each monitoring one or more patients. The sleep sessions' information enables one or more caregivers to analyze, diagnose and/or recommend a treatment to the one or more patients. Priority may be assigned to the plurality of sleep breathing disorder events so as to allow the one or more caregivers to respond in orderly manner. The caregivers may be alerted and/or notified of events relating to sleep breathing disorder events of the plurality of patients. Prioritization, notification and/or alert may be done according to pre-defined rules which may be adjusted by the caregiver, for example, severity and/or patient history.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire line, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

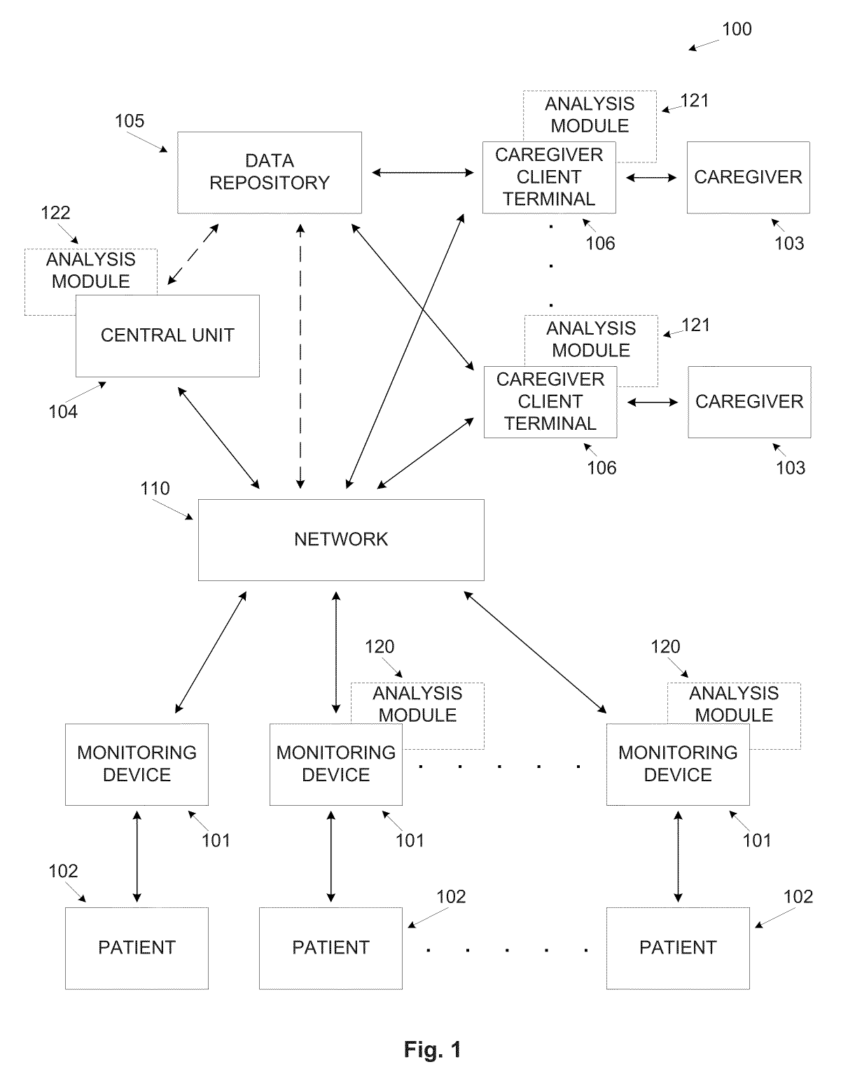

Reference is now made to FIG. 1 which is a schematic illustration of an exemplary system for monitoring and managing sleep breathing disorders, according to some embodiments of the present invention. A sleep breathing disorder management system 100 includes a plurality of monitoring devices 101, each monitoring one or more patients 102. The monitoring device 101 may be a handheld client terminal and may be facilitated through existing devices for example, Smartphone, tablet, laptop computers and/or through a custom monitoring device. The monitoring device 101 connects to one or more sensors, for example, microphone, heart beat rate monitor, pulse oximeter and/or movement sensor which monitor the patient 102. The sensors may be integrated in the monitoring device 101 and/or external to the monitoring device 101. In case the sensor is external to monitoring device 101, the sensor may communicate with the monitoring device 101 through one or more wired and/or wireless interconnections. Wired connection may include, for example, serial I/O, universal serial bus (USB) and/or Ethernet. Wireless connection may include, for example, wireless local area network (WLAN), Bluetooth, infra red (IR) and/or radio frequency (RF).

The monitoring device 101 collects the monitoring information from the sensors and analyzes the monitoring information to create one or more patterns of the vital life signs. The monitoring device 101 may execute an analysis software module 120 to analyze the patterns to identify one or more sleep breathing disorder events. The monitoring device 101 communicates with one or more caregivers 103 using a client terminal 106, for example, Smartphone, tablet, laptop computers, desktop computer and/or work station over one or more networks 110, for example, cellular and/or internet. The monitoring device 101 may transmit the raw monitoring information and/or the processed patterns to the caregiver 103 for diagnosis and recommendation for treatment. The caregiver 103 has access to the patterns transmitted from the monitoring device 101 through a client application executed on the client terminal 106. The client terminal 106 used by the caregiver 103 may include a data repository 105 which stores history records of the monitoring information of the patients 102. The history records may be available to the caregiver 103 using the client terminal. Using the client terminal(s) 106, the caregiver(s) 103 may access the information received from the monitoring device 101 at any time, either in real time or offline at a later time.

Optionally, alerts are generated to indicate the caregiver 103 of a sleep breathing disorder event. A plurality of alert rules may be created and/or adjusted to define one or more condition(s) for generating the alert. The alert rules may be automatically set using default values and/or set according to past sleep breathing disorder events of the patient 102.

Optionally, a central unit 104 which connects to the network 110 manages the communication between the monitoring devices 101 and the client terminals 106 used by the caregivers 103. The central unit 104, for example, server, cluster of processing nodes and/or cloud service may receive the patterns from the monitoring devices 101 and analyze them to generate the alerts to the caregivers 103. The central unit may connect to the data repository unit 105, for example, server and/or cloud service which may store history records of the monitoring information of the patients 102. The data repository 105 may connect directly to the central unit 104 or it may communicate with the central unit 104 over the network 110.

Optionally, the central unit 104 receives the monitoring information from the monitoring device 101 and executes an analysis software module 122 to analyze the monitoring information and identify sleep breathing disorder events. The monitored data and/or the analysis to the caregiver 103 using the client terminal 106.

Optionally, one or more of the client terminals 106 receives the monitoring information from the monitoring device 101 and executes an analysis software module 121 to analyze the monitoring information and identify sleep breathing disorder events. The monitored data and/or the analysis to the caregiver 103 using the client terminal 106.

Optionally, processing and/or analysis of the monitoring information are distributed between one or more of the analysis modules 120, 121 and/or 122.

Optionally, the caregivers 103 using the client terminal 106 have access to the data repository 105 over the network 110 to get access to the history records of the monitoring information of the patients 102.

Optionally, the caregivers 103 using have access to the patterns through web based service accessible by a web browser executed on the client terminal 106.

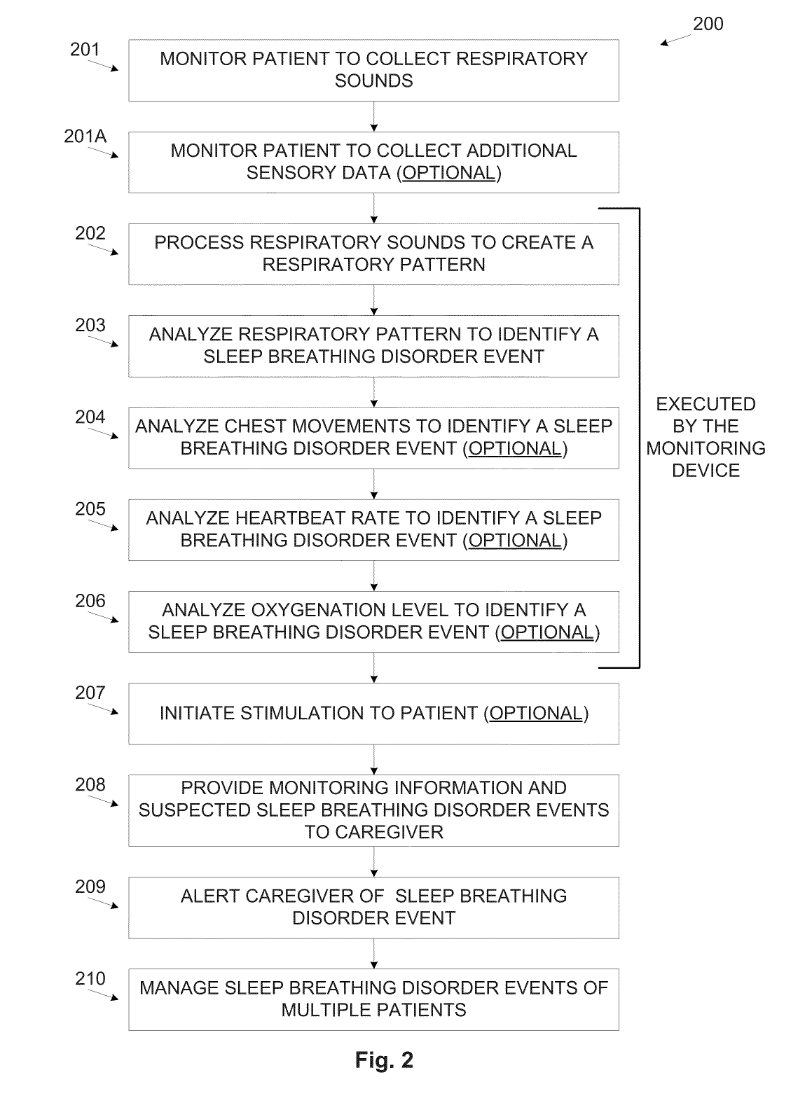

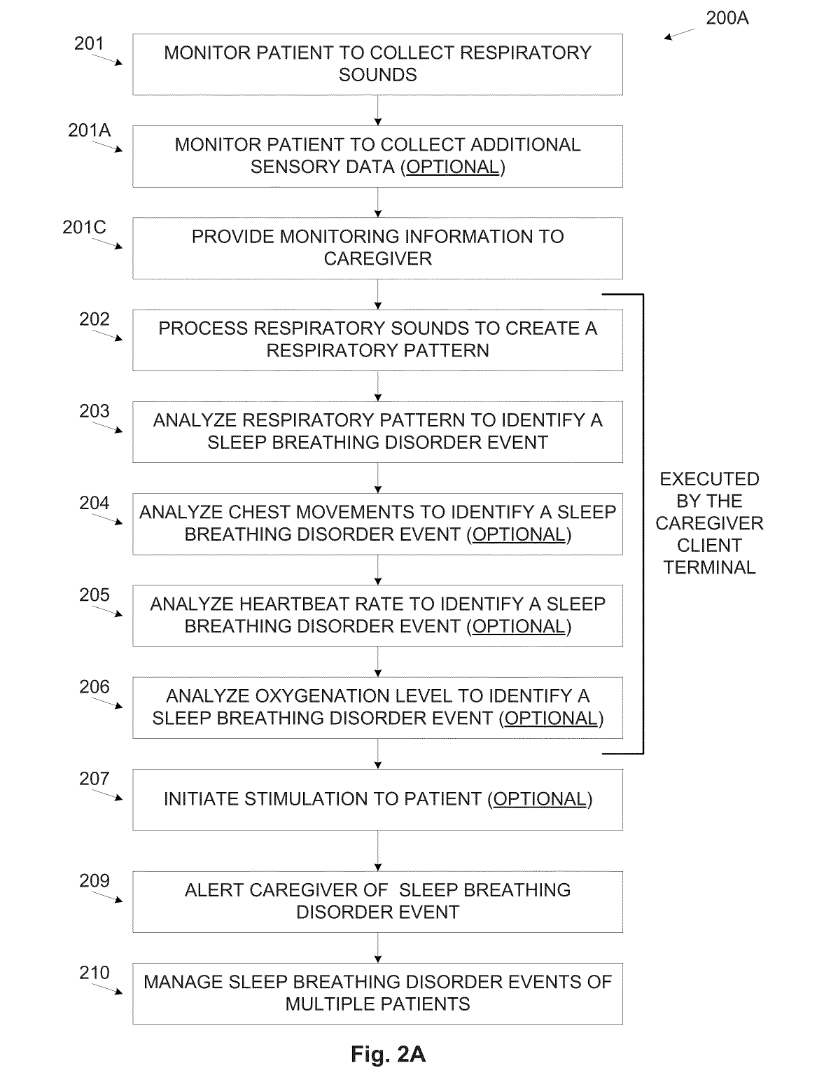

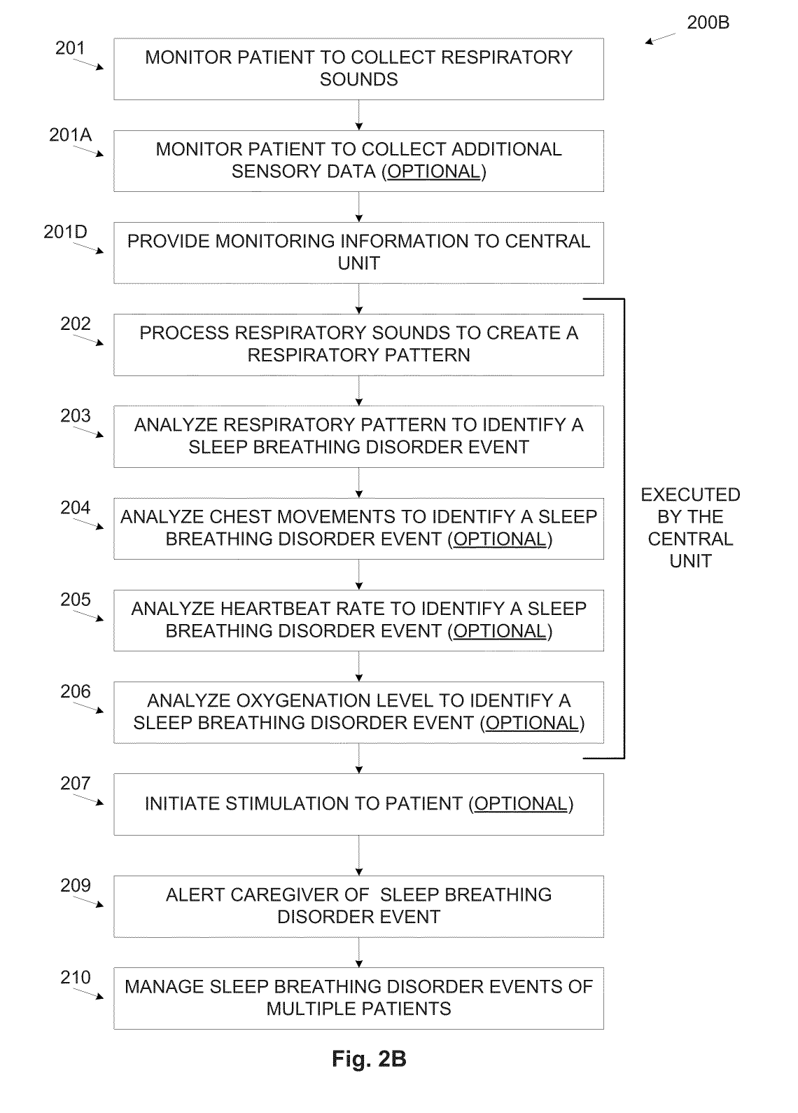

Reference is now made to FIG. 2 which is a flowchart of an exemplary process of monitoring and managing sleep breathing disorder events in which processing and analysis is performed at the monitoring device, according to some embodiments of the present invention. As shown at 201, a process 200 which may be utilized through the exemplary system 100 starts with collecting the respiratory sounds of the patient 102 using a sensor, for example a microphone. Monitoring information may include additional vital life signs, for example, heart beat rate, oxygenation and/or movement. However, the respiratory sound is the most evident, deterministic and simple to measure symptom of the sleep breathing disorder.

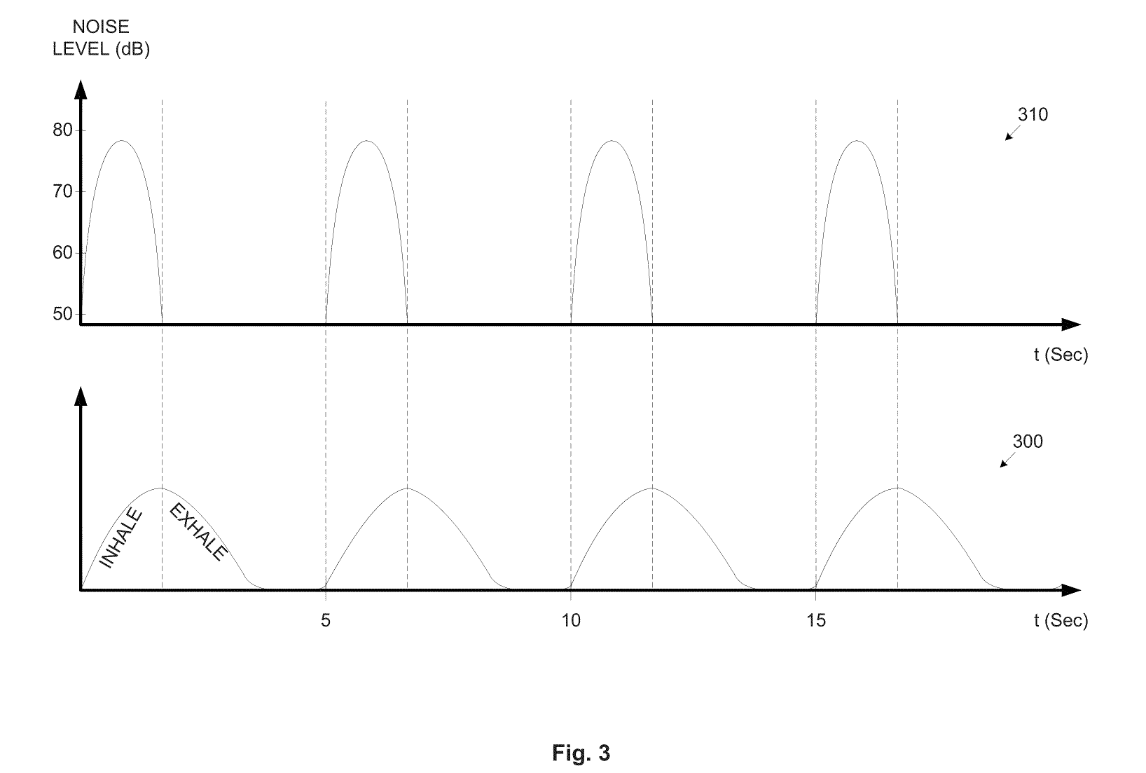

Reference is now made to FIG. 3 which is a schematic illustration of a typical breathing cycle and an exemplary excessive respiratory sound level cycle. A typical breathing cycle 300 includes an inhalation (inspiration) phase of approximately 1.5-2 seconds, an exhalation (expiration) phase of approximately 1.5-2 seconds and a natural pause of breathing of approximately 2 seconds. An exemplary respiratory sound level graph 310 represents an exemplary respiratory sound level with respect to the breathing cycle 300. In most cases of sleep breathing disorders, excessive respiratory sound levels are recorded during the inhalation phase. As is shown through the respiratory sound level graph 310 the sound level which may result from, for example, snoring and/or wheezing reaches high volume levels during the inhalation phase.

Reference is now made once again to FIG. 2. Optionally, as shown at 201A, additional sensors are used to collect additional sensory data (vital life signs), for example, heart beat rate, oxygenation in the blood stream and/or chest movement which are indicative of sleep disorder events. The one or more sensors may be integrated in the monitoring device 101 and/or external to the monitoring device 101. In case the sensor is external to monitoring device 101, the sensor communicates with the monitoring device 101 through wired and/or wireless interconnection. Wired connection may include, for example, serial I/O, USB and/or Ethernet. Wireless connection may include, for example, WLAN, Bluetooth, IR and/or RF. The monitoring information collected from the one or m more sensor may be processed and analyzed separately by the monitoring device 101 and/or processed with respect to each other to provide a more accurate analysis and identification of sleep breathing disorder events.

As shown at 202, the monitoring device 101 processes the collected respiratory sounds in real time to identify the pattern of the respiratory cycle over time. Processing the respiratory sounds is required to identify the waveform of the respiratory cycle. The respiratory sounds are processed with respect to their amplitude while their frequency is not evaluated and is not used for processing the respiratory sounds. The respiratory cycle may consist of a plurality of sound amplitudes at a plurality of levels thus creating a jugged and/or non-coherent presentation of the respiratory cycle. Local drops or rises of the amplitudes may be captured which may further obscure the presentation of the respiratory cycle. In addition, environment noise may be present that obscures the respiratory sounds and may be distorted and/or degraded. To create a coherent presentation of the respiratory cycle the amplitudes are processed using several techniques.

Processing the amplitudes of the respiratory sounds is done by creating an envelope waveform that encapsulates the plurality of amplitudes to create a coherent waveform depicting the respiratory cycle in order to identify one or more sleep breathing disorder events and/or avoid false detection of such events. The maximum and/or minimum values of the amplitudes are identified and interpolation, averaging and/or prediction are employed to construct the envelope waveform representing the respiratory cycle. Previous time periods of the respiratory cycle which are stored may be used for better processing and construction of the envelope waveform.

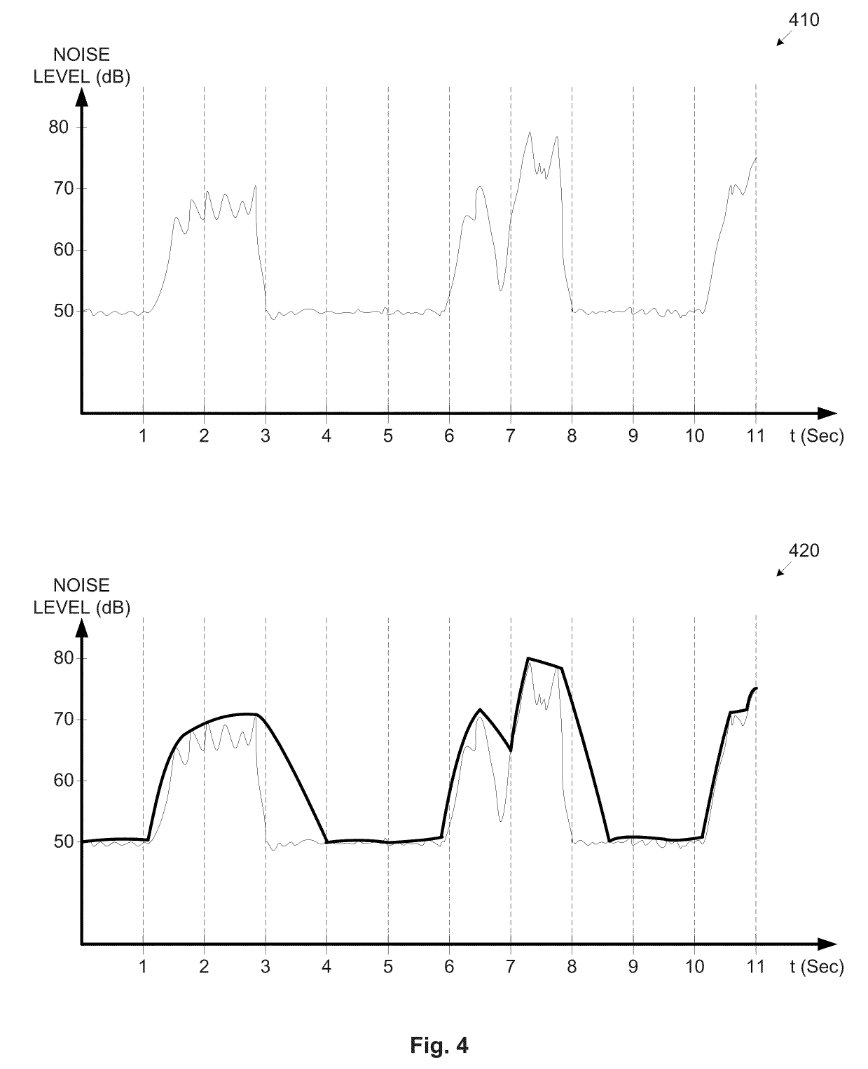

Reference is now made to FIG. 4 which is a schematic illustration of an exemplary respiratory cycle construction, according to some embodiments of the present invention. An exemplary respiration sound cycle 410 which was captured by the sensor includes a plurality of amplitudes presenting a jugged waveform with local rises and falls presenting a problem for properly extracting the respiratory cycle. As shown by the graph 420, an envelope (denoted by the thick line) is created that encapsulates the amplitudes providing a clear presentation of the respiration cycle. As is seen for this exemplary respiratory cycle typical ambient noise may be approximately 50 dB.

Optionally, dynamic threshold is set while monitoring the respiratory sounds. Ambient and/or environment noise may be present in the environment in which the patient 102 is monitored. Environment noise may include monotonous noise, for example, FAN and/or air conditioning. A dynamic threshold may be set to be slightly above the environment noise in order reduce the effect of the environment noise to more accurately extract the respiratory sounds while monitoring the patient 102 and/or to avoid false identification of sleep breathing disorder events. The threshold level may be low enough so it may effectively capture the respiratory sounds, however it may not be too low so as to interpret normal breathing as excessive respiratory sounds, such as snoring and/or wheezing.

The algorithm for dynamic threshold adjustment sets a default threshold level. The noise of the environment which may include respiratory sound is continuously recorded while monitoring the patient 102 and is averaged. The averaged environment noise level is compared with the threshold level and in case the averaged environment noise level exceeds the threshold by a pre-defined value the threshold level may be adjusted to equal the averaged environment noise. The threshold may be set slightly higher than the averaged environment noise level in order to avoid identification of normal respiratory sound as excessive respiratory sound and mark them as candidates for an apnea or hypopnea event.

A numeric example is provided herein to illustrate the dynamic threshold adjustment algorithm. The default threshold is set to 45 dB. The minimum threshold level is set to 42 dB. These values are exemplary values which are computed for home environment. The threshold level may need to be calibrated for the monitoring device and/or sensors which are used for monitoring the patient 102. The environment noise is continuously recorded and averaged. The averaged environment noise level is then added with, for example, 3 dB to have the threshold set to a slightly higher level than the environment noise. The averaged environment noise is compared with the current threshold level, for example the default threshold level of 45 dB. In case the averaged environment noise level is higher or lower than the current threshold by a pre-defined value, for example, 3 dB, the threshold is set to a new level which is slightly higher from the newly computed value of the averaged environment noise.

Optionally, a microphone fading algorithm is employed to reduce microphone effects, for example, decaying and/or fading which may distort the captured respiratory sounds amplitudes. Using such an algorithm enables the use of standard microphones which are integrated in the monitoring device 101 which utilizes available devices, such as, Smartphone, tablet and/or laptop computer. These standard microphones and/or other audio devices used for sampling the respiratory sounds may be susceptible to decaying and/or fading effects. The fading effects may be present following the last amplitude peak of the captured respiratory sounds. The goal of the algorithm is to compensate for the fading effects and reconstruct the actual time period of the wave and the silent time period between two consecutive waves. The algorithm is based on computing a corrected fall time of the wave by employing a time correction constant which is characteristic to the microphone in use. The difference between the peak of the wave and the threshold is measured and multiplied by characteristic time constant and the outcome is the corrected fall time of the wave. The corrected fall time is compared with the actual fall time of the wave and the smaller value of the two is selected to be the fall time which is used for analyzing the respiratory sounds cycle to identify a sleep breathing disorder event. The computation may require time stamps of previous waves are used. The parameters used for the computation, for example, time correction constant, may need to be adjusted per microphone and/or per monitoring device. An exemplary implementation of the algorithm for reducing the fading effects is depicted in Pseudo Code Excerpt 1 below.

### Pseudo Code Excerpt 1:

### Variables:

TR denotes the threshold level.
H denotes the hysteresis factor. Hysteresis is required to prevent the algorithm from fluctuating between states.
LPT denotes the time stamp of the last peak of the wave.
LPV denotes the volume of the last peak of the wave.
EST denotes the time stamp of the wave end (when the volume goes below the threshold level deducted with the hysteresis factor).
TD denotes the wave fall time or fading time.
MD denotes the maximum distance of the wave peak above the threshold (with the hysteresis factor deducted from it).
CC denotes the correction constant which is calibrated per microphone and/or monitoring device.
TC denotes the time correction factor.
ST denotes the measured time period of the wave duration.
STC denotes the corrected time period of the wave duration.

### Algorithm:

### 1) TD=LPT−EST;

### 2) MD=LPV−(TR−H);

### 3) TC=CC*MD;

### 4) IF (TC<TD) THEN STC=ST−TC;

### 5) ELSE STC=ST−TD;

### Notes:

(1) If TC or TD are deducted from ST they need to be added to the silent time period between the wave and the succeeding wave.

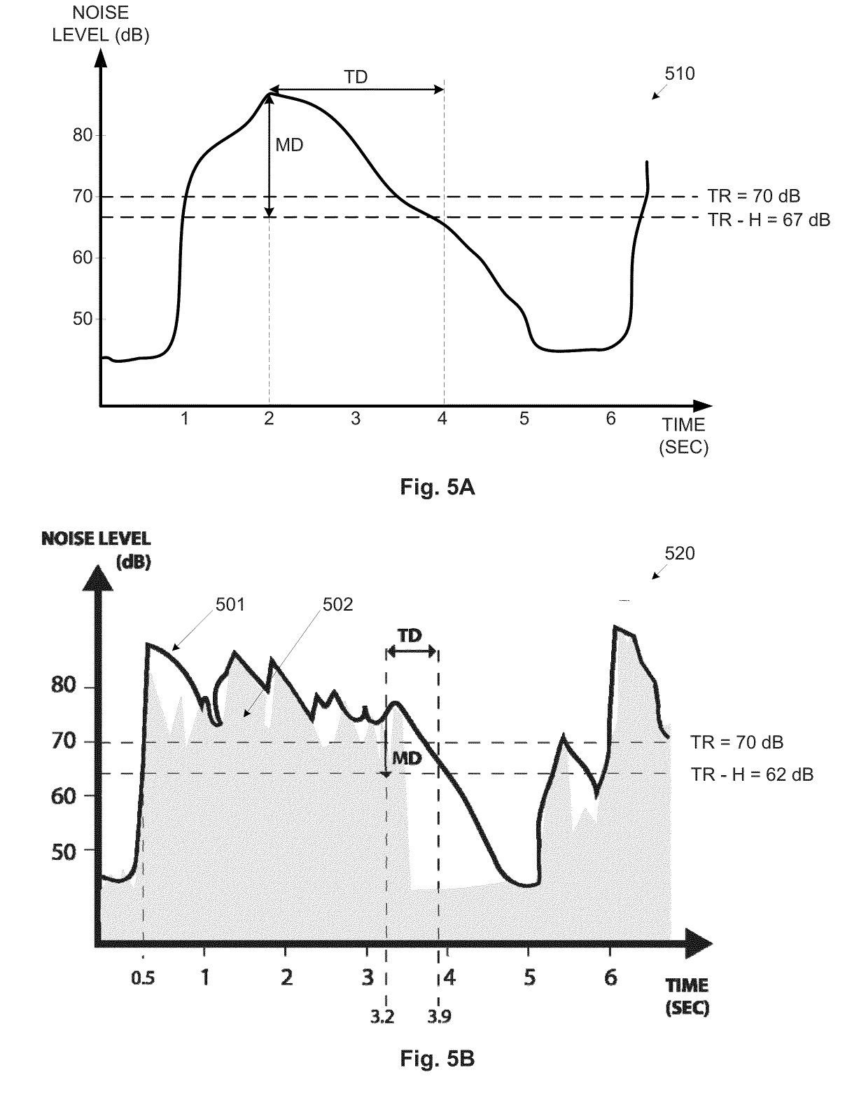

Reference is now made to FIG. 5A which is a schematic illustration of an exemplary computation for reducing microphone fading effect, according to some embodiments of the present invention. A waveform 510 presents an exemplary wave captured and processed for identifying sleep disorder events. The vertical axis represents the sound volume and the horizontal axis represents time. In the exemplary fading effects reduction, the following values are given:

TR=70 dB, H=3 dB, LPT=4 sec, EST=2 sec, LPV=85 dB, CC=0.08, ST=4−1=3 sec;
1) TD=LPT−EST=4−2=2 sec;

### 2) MD=LPV−(TR−H)=85−(70−3)=18 dB;

### 3) TC=CC*D=0.08*18=1.44;

### 4) (TC<TD) THEN

STC=ST−TC=3−1.44=1.56 sec;

Therefore for this example, the corrected wave time period (duration) is set to 1.56 sec and not 3 sec as is evident from the waveform graph 510. As noted the TC time period (1.44 sec) is added to the silent time period following the wave.

Reference is now made to FIG. 5B which is a schematic illustration of an exemplary computation for reducing microphone fading effect for a processed respiratory cycle, according to some embodiments of the present invention. A waveform graph 520 presents an exemplary respiratory sound cycle which is processed to create an envelope waveform 501 that encapsulates the plurality of amplitudes 502 to create a distinct waveform 501 depicting the respiratory cycle to identify sleep disorder events. The vertical axis represents the sound volume and the horizontal axis represents time. It is noted that some aspects in the graph 520 are distorted to allow understanding of the graph 520, for example, the amplitudes 502 are stretched high, the respiratory sound time period is stretched to approximately 3.5 sec (typically it is 2 sec) and the natural pause between two adjacent respiratory sounds is squeezed (typically it is approximately twice the time period of the respiratory sound wave).

In the exemplary fading effects reduction, the following values are given:
TR=70 dB, H=8 dB, LPT=3.9 sec, EST=3.2 sec, LPV=75 dB,
CC=0.08, ST=3.9−0.5=3.4 sec;
1) TD=LPT−EST=3.9−3.2=0.7 sec;

### 2) MD=LPV−(TR−H)=75−(70−8)=13 dB;

### 3) TC=CC*MD=0.08*13=1.04;

### 4) (TC>TD) THEN

STC=ST−TD=3.4−0.7=2.7 sec;

Therefore for this example, the corrected wave time period (duration) is set to 2.7 sec and not 3.4 sec as is evident from the waveform graph 520. As noted the TD time period (0.7 sec) is added to the silent time period following the wave.

Reference is now made once again to FIG. 2. As shown at 203, the pattern extracted from the respiratory sounds amplitudes is analyzed to identify irregularities in the respiratory cycle which may be suspected as sleep breathing disorder events, for example, apnea and/or hypopnea.

The pattern is continuously analyzed to identify the start and end points of each wave in the pattern are identified, where wave refers to rise and fall cycle of sound within the processed waveform. The analysis also marks the top (summits) point level and the bottom (nadirs) point level of each wave. In case the bottom level of the wave is below the environment noise level, the level of the bottom point is considered to be the environment noise level. The time width of each wave is calculated.

Waves which are too long or too short to be considered as excessive respiratory sounds are disqualified. Waves which do not keep a minimum natural pause phase between each other are disqualified.

Each wave is compared to a preceding waves and the wave is qualified as an excessive respiratory sound in case there is a sequence of pre-defined number of waves preceding the wave.

In case a pre-defined silent time period (i.e. falls between minimum and/or maximum time limits) is identified between adjacent waves it is marked as a candidate for an apnea event. The silent time period is pre-defined with default minimum and/or maximum time values and may be manually adjusted. In case during the silent time period identified between adjacent waves, shallow waves are identified, the time period is marked as candidate for a hypopnea event.

An exemplary algorithm is described in pseudo code excerpt 2 which describes computation of probability for a sleep disorder event based on processing the recorded respiratory sounds level. The algorithm is based on searching the respiratory sounds level wave form for patterns in which there are excessive respiratory sounds level followed by silence and/or low respiratory sounds level followed again by excessive respiratory sounds level. Such a pattern is suspected as being a candidate for an apnea or hypopnea event. Note that this scenario may be ignored in case the pattern is preceded by a stimulation initiated to the patient 102 to cause the patient 102 to exit the sleep breathing disorder event as described hereinafter.

### Pseudo Code Excerpt 2:

### Variables:

X denotes the computed probability of a candidate for apnea or hypopnea event being a real apnea or hypopnea event (0≦X≦1).
Z denotes a predefined minimum probability to be pursued during the probability computation.
T denotes the duration of the silent time period (seconds).
T1 denotes a predefined maximum duration of the silent time period (seconds).
T2 denotes a predefined minimum duration of the silent time period (seconds).
S denotes the peak (maximum) linear respiratory sounds level during the silent time period.
S0 denotes the minimum linear respiratory sounds level during the silent time period.
M denotes the peak (maximum) linear respiratory sounds level during the period preceding the silent time period.
N denotes the peak (maximum) linear respiratory sounds level during the period succeeding the silent time period.
A denotes the maximum respiratory sounds level difference from M.
B denotes the maximum respiratory sounds level difference from N.
C1 denotes the weight (importance) assigned to deviation of A from M.
C2 denotes the weight (importance) assigned to deviation of B from N.

### Algorithm:

### 1) X=0;

### 2) IF (S<S0) THEN S=S0;

### 3) IF [(S≧M) OR (S≧N)] THEN EXIT;

### 4) IF [(T<T1) OR (T>T2)] THEN

### 5) X=[SQRT(Min (M−S, A))*C1]+[SQRT(Min (N−S, B))*C2]

### 6) IF (X<Z) THEN X=0;

### 7) ELSE IF (X>1) THEN X=1;

### Notes:

(1) The algorithm is designed to operate over a linear scale of respiratory sounds level between 40 dB and 100 dB. Respiratory sounds level which is below 40 dB is mapped to 40 dB and respiratory sounds level which is above 100 dB is mapped to 100 dB. Linearity is maintained by converting the respiratory sounds level expressed in dB using the following conversion formula:
   L1=(L−40)/6L denotes the respiratory sounds level as recorded expressed in dB.
   LL denotes the respiratory sounds level after the linear conversion.
      The outcome of the algorithm provides the value of X which is the computed probability of the candidate for apnea or hypopnea event to be a real apnea or hypopnea event. In case the value of X is 0 for a specific candidate for apnea or hypopnea event then no attempt is done to improve the probability rate and the specific candidate is not considered as a real event.

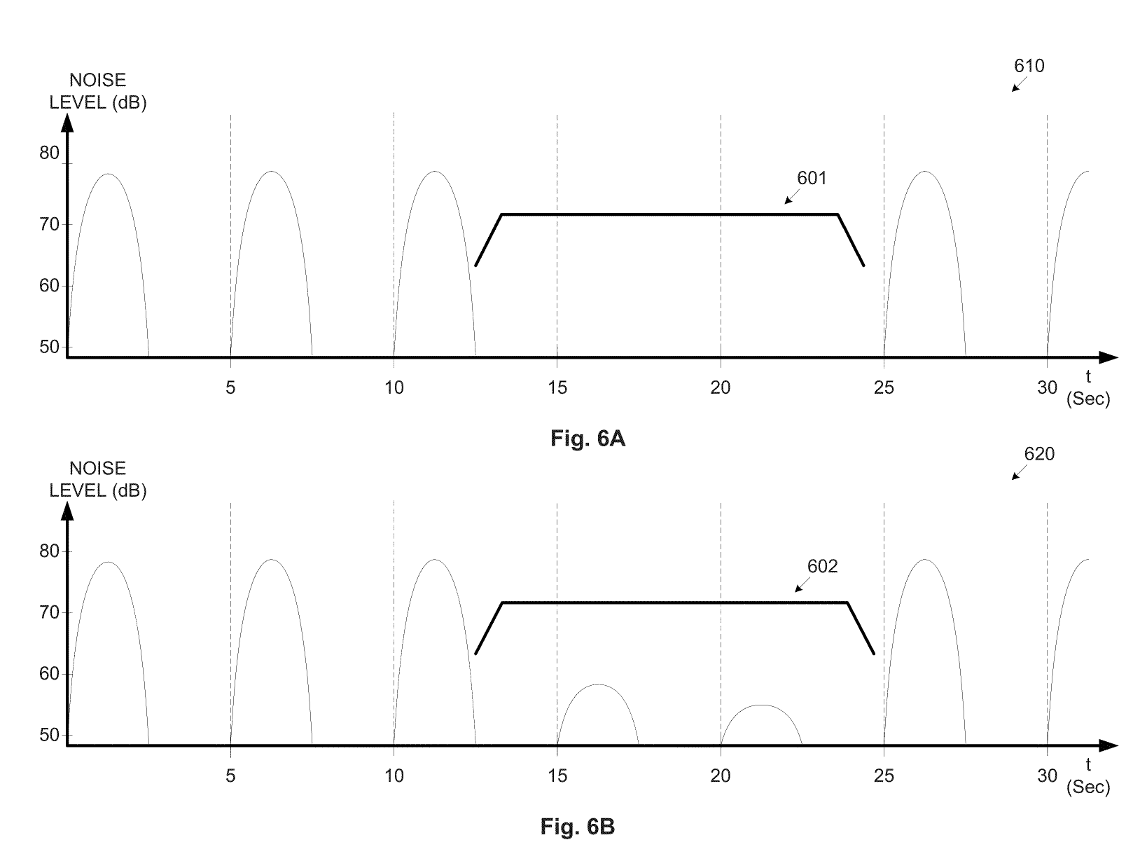

Reference is now made to FIG. 6A which is a schematic illustration of identifying a suspected apnea event during an exemplary respiratory cycle containing snoring and/or wheezing sounds, according to some embodiments of the present invention. An exemplary respiration sound cycle 610 which was captured and processed by the monitoring device includes a plurality of waves representing a respiratory cycle. A time period 601 is marked as candidate for an apnea event since a silent time period is identified between time stamp 12 and time stamp 25. After marking the time period 601 as candidate for a suspected apnea event, additional succeeding waves may be identified and analyzed to improve a probability that the time period 601 is a suspected apnea event. The computation of the probability is described hereinafter.

Reference is now made to FIG. 6B which is a schematic illustration of identifying a suspected hypopnea event during an exemplary respiratory cycle containing snoring and/or wheezing sounds, according to some embodiments of the present invention. An exemplary respiration sound cycle 620 which was captured and processed by the monitoring device includes a plurality of waves representing a respiratory cycle. A time period 602 is marked since candidate for a hypopnea event as a time period containing shallow waves (interpreted as hypopnea) is identified between time stamp 12 and time stamp 25. After marking the time period 602 as candidate for a hypopnea event, additional succeeding waves are identified and analyzed to improve the probability that the time period 502 is a suspected hypopnea event.

Optionally, stimulation is initiated to the patient 102 when a sleep breathing disorder event is identified. The stimulation is initiated by the monitoring device 101 to intervene with the sleep session of the patient 102 in order to cause the patient 102 to exit the sleep disorder event with the intension not to bring the patient 102 to full arousal. Stimulation is done using a stimulation device, for example, audio stimulation and/or tactile intervention. The stimulation device, for example, audio output device, vibration device and/or may be integrated within the monitoring device 101 and/or external to the monitoring device 101. In case the stimulation device is external to monitoring device 101, the stimulation device communicates with the monitoring device 101 through wired and/or wireless interconnection. Wired connection may include, for example, serial I/O, USB and/or Ethernet. Wireless connection may include, for example, WLAN, Bluetooth, IR and/or RF. Records of stimulation events may be transmitted to the one or more caregivers 103.

Optionally, in case additional sensory data was monitored and collected, the additional sensory data may be processed and/or analyzed to increase accuracy in identifying suspected sleep disorder candidates.

Monitoring the chest movement of the patient 102 may be done using one or more monitoring device, for example, movement sensors, accelerometer and/or gyro. One or more movement monitoring devices may be attached to the patient 102, for example, to the arm and/or to the upper chest. The intensity of chest movement may be indicative of sleep breathing disorder events as the chest movements may continue and/or increase temporarily during and immediately after a sleep breathing disorder event as the chest of the patient 102 may attempt to overcome the blockage in the airways. Body posture information of the patient 102 may be collected using position sensors in order to better evaluate the chest movements of the patient 102 with respect to the body posture of the patient 102.

Reference is now made once again to FIG. 2. Optionally, as shown at 204, when the chest movements of the patient 102 are monitored, a probability computation based on chest movement analysis may be performed to improve and/or validate the probability computed based on the respiratory sounds level using the algorithm described in the pseudo code excerpt 2 above.

Posture monitoring may further improve the probability computation performed based on analysis of the chest movements. Excessive posture shifts which may indicate the patient 102 is shifting sleeping positions may be used to disqualify suspected sleep breathing disorder candidates by screening sleep breathing disorder candidates during which excessive posture changes was identified using a simple screening criterion. An exemplary algorithm is described in pseudo code excerpt 3 which describes further computation of probability for a sleep disorder event based on processing the monitored chest movements. Employing the algorithm described in the pseudo code excerpt 3 is performed to improve and/or validate X in case the value of X is greater than Z following the probability computation made using the algorithm based on analysis of the respiratory sounds as described in the pseudo code excerpt 2.

### Pseudo Code Excerpt 3:

### Variables:

P0 denotes the peak (maximum) level of the chest movements during the silent time period.
P1 denotes the peak (maximum) level of the chest movements preceding the silent time period.
P2 denotes the peak (maximum) level of the chest movements succeeding the silent time period.
C3 denotes the weight (importance) assigned to deviation of P1 from P0.
C4 denotes the weight (importance) assigned to deviation of P2 from P0.

### Algorithm:

### 1) IF (P1>P0) AND (P2>P0) THEN

### 2) X=X+{(1−X)*[(SQRT(P1−P0)*C3)+(SQRT(P2−P0)*C4)]}

### 3) IF (X>1) THEN X=1;

### Notes:

(1) The analysis of the chest movements and the analysis of the respiratory sounds are synchronized in time so as to have the two analyses indicate the same time period in which the candidate for apnea or hypopnea event takes place. Minor shifts in time between the analysis of the monitored chest movements and the respiratory sounds may be applied as the two symptoms may typically lag one after the other.
(2) Chest movement analysis is performed using data received from a movement measuring device, for example, accelerometer and/or gyroscope. During calculation the recorded movements' values are converted to a linear scale in the range of 0 through 10. Movement values above 10 are considered noise resulting from posture change or other excessive movements.
(3) In case a posture change is identified just before the time period in which a specific candidate for apnea or hypopnea takes place, the algorithm ignores the specific candidate and does not try to improve probability. This means the specific candidate is not considered as a real apnea or hypopnea event from the chest movements' analysis perspective.
(4) The chest movement analysis may be performed prior to the respiratory sounds analysis in order to immediately disqualify (ignore) candidates for apnea or hypopnea which are the result of a posture shift of the patient 102 as identified during the chest movement analysis.

Monitoring the heart beat rate of the patient 102 may be done using one or more monitoring devices, for example, conventional heart beat rate monitor and/or pulse oximeter. Heartbeat rate may be expressed using beats per minute (BPM). The heartbeat rate may be indicative of sleep breathing disorder events as the heart beat rate is increased during and immediately after a sleep breathing disorder event as result of the effort required to overcome the blockage in the airways which may be the cause for the sleep breathing disorder event.

Optionally, as shown at 205, when the heartbeat rate of the patient 102 is monitored, a probability computation based on heartbeat rate (BPM) may be performed to improve and/or validate the previously computed probability based on respiratory sounds and/or chest movements as discussed hereinabove.

An exemplary algorithm is described in pseudo code excerpt 4 which describes further computation of probability for a sleep disorder event based on processing the heartbeat rate. Employing the algorithm described in the pseudo code excerpt 4 is performed to improve and/or validate X in case the value of X is greater than Z following the probability computation made using the previously described algorithms based on analysis of the respiratory sounds and/or chest movements. The computation made for analysis of the respiratory sounds and the chest movements are described in the pseudo code excerpts 2 and 3 respectively.

### Pseudo Code Excerpt 4:

### Variables:

B0 denotes the peak (maximum) level of heartbeat rate during the silent time period.
B1 denotes the peak (maximum) level of the heartbeat rate preceding the silent time period.
B2 denotes the peak (maximum) level of the heartbeat rate succeeding the silent time period.
C5 denotes the weight (importance) assigned to deviation of B1 from B0.
C6 denotes the weight (importance) assigned to deviation of B2 from B0.

### Algorithm:

### 1) IF (B1>B0) AND (B2>B0) THEN

### 2) X=X+{(1−X)*[(SQRT(B1−B0)*C5)+(SQRT(B2−B0)*C6)]}

### 3) IF (X>1) THEN X=1;

### Notes:

(1) The analysis of the heartbeat rate and the analysis of the respiratory sounds are synchronized in time so as to have the two analyses indicate the same time period in which the candidate for apnea or hypopnea event takes place. Minor shifts in time between the analysis of the monitored heartbeat rate and the respiratory sounds may be applied as the two symptoms may typically lag one after the other.

Monitoring the level of oxygenation (oxygen saturation) in the blood stream of the patient 102 may be done using one or more monitoring device, for example, pulse oximeter. The level of oxygenation in the blood stream may be indicative of sleep breathing disorder events as the oxygenation level may be temporarily reduced during and immediately after a sleep breathing disorder event as result of the temporarily lack of air in the lungs which may be caused by the sleep breathing disorder event.

Optionally, as shown at 206, when the oxygenation level in the blood of the patient 102 is monitored, a probability computation based on oxygenation level may be performed to improve and/or validate the previously computed probability based on respiratory sounds, chest movements and/or heartbeat rate as discussed hereinabove.

An exemplary algorithm is described in pseudo code excerpt 5 which describes further computation of probability for a sleep disorder event based on processing the heartbeat rate. Employing the algorithm described in the pseudo code excerpt 5 is performed to improve and/or validate X in case the value of X is greater than Z following the probability computation made using the previously described algorithms based on analysis of the respiratory sounds, chest movements and/or heartbeat rate. The computation made for analysis of the respiratory sounds, the chest movements and heartbeat rate are described in the pseudo code excerpts 2, 3 and 4 respectively.

### Pseudo Code Excerpt 5:

### Variables:

T3 denotes the predefined time after the suspected apnea/hypopnea event during which we measure for a decrease in blood oxygenation.
G0 denotes the peak (maximum) level of oxygen saturation in the blood during the silent time period.
G1 denotes the peak (maximum) level of the oxygen saturation in the blood preceding the silent time period.
G2 denotes the valley (minimum) level of the oxygen saturation in the blood succeeding the silent time period (within the T3 period).
C7 denotes the weight (importance) assigned to deviation of G1 from G0.
C8 denotes the weight (importance) assigned to deviation of G2 from G0.

### Algorithm:

### 1) IF (G1>G0) AND (G2>G0) THEN

### 2) X=X+{(1−X)*[(SQRT(G1−G0)*C7)+(SQRT(G2−G0)*C8)]}

### 3) IF (X>1) THEN X=1;

### Notes:

(1) The analysis of the oxygenation level and the analysis of the respiratory sounds are synchronized in time so as to have the two analyses indicate the same time period in which the candidate for apnea or hypopnea event takes place. Shifts in time between the analysis of the monitored oxygen level and the respiratory sounds may be applied as the two symptoms may typically lag one after the other (a decrease in the oxygenation level will typically lag by up to a minute after an apnea event).

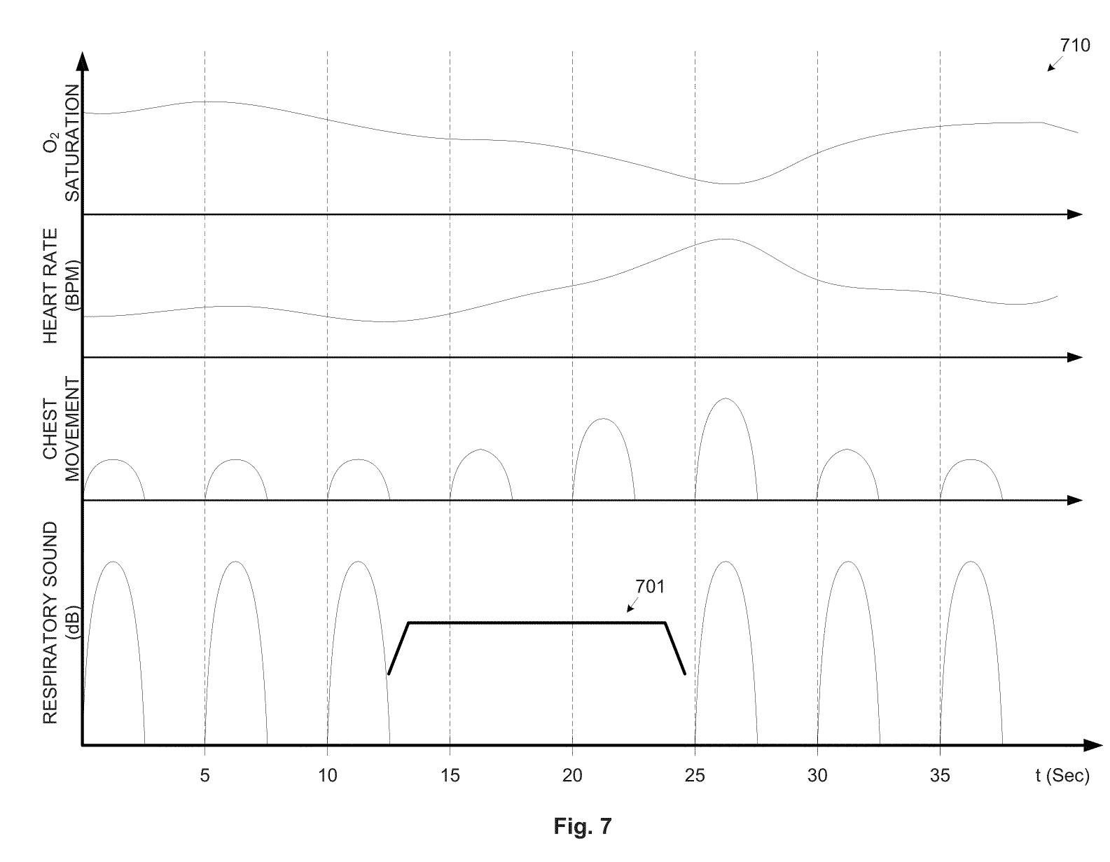

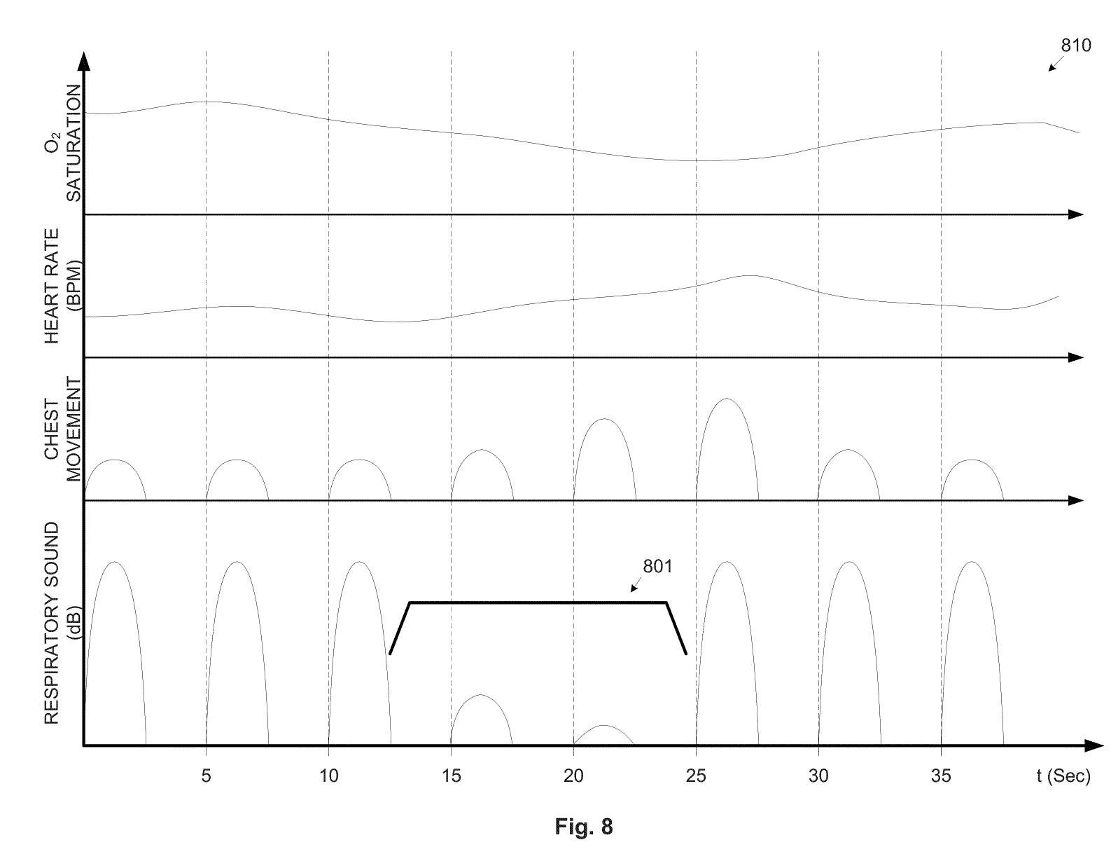

Reference is now made to FIG. 7 which is a schematic illustration of identifying a suspected apnea event using multiple sensors, according to some embodiments of the present invention. An exemplary graph 710 presents monitoring information captured from a plurality of sensor and processed by the monitoring device 101, including, respiratory sound, chest movement, heart beat rate and oxygenation level in the blood stream. A time period 701 is marked as candidate for an apnea event since a silent time period is identified between time stamp 12 and time stamp 25. The time period 701 is further suspected as a candidate for an apnea event as the chest movements are slightly increased, the heart beat rate is increased and the oxygenation level is reduced during the time period 701. After marking the time period 701 as candidate for an apnea, the captured monitoring information presents additional succeeding respiratory sound waves, increased chest movements, increased heart beat rate and reduced oxygenation level immediately following the time period 701 and the time period 701 is marked as a suspected apnea event.

Reference is now made to FIG. 8 which is a schematic illustration of identifying a suspected hypopnea event using multiple sensors, according to some embodiments of the present invention. An exemplary graph 810 presents monitoring information captured from a plurality of sensor and processed by the monitoring device 101, including, respiratory sound, chest movement, heart beat rate and oxygenation level in the blood stream. A time period 801 is marked as candidate for a hypopnea event since a silent time period is identified between time stamp 12 and time stamp 25. The time period 801 is further suspected as a candidate for a hypopnea event as the chest movements are slightly increased, the heart beat rate is increased and the oxygenation level is reduced during the time period 801. After marking the time period 801 as candidate for an apnea, the captured monitoring information presents additional succeeding respiratory sound waves, increased chest movements, increased heart beat rate and reduced oxygenation level immediately following the time period 801 and the time period 801 is marked as a suspected hypopnea event.

Optionally, as shown at 207, in the event of identifying a suspected sleep breathing disorder, stimulation is initiated to the patient 102 by the monitoring device 101 through a stimulation device. The stimulation is provided to intervene with the sleep session of the patient 102 in order to cause the patient 102 to exit the sleep disorder event with the intension not to bring the patient 102 to full arousal.

Reference is now made once again to FIG. 2. As shown at 208, the sleep session information, for example, pattern of the respiratory sounds, suspected sleep breathing disorder events, monitoring data from additional sensors and/or stimulation events, is transmitted to one or more caregivers 103 for diagnosis and recommendation for treatment. The monitoring information and/or part of it may be transmitted in real time as it is collected, processed and analyzed by the monitoring device 101 and/or it may be transmitted after the patient 102 has completed the sleep session.

Optionally, monitoring information is transmitted to the one or more caregivers 103 after the patient 102 confirms the transmission.

Optionally, in the event the monitoring device 101 does not have connection to the network 110 during and/or after the sleep session, the monitoring information is sent to the one or more caregivers 103 once the monitoring device gains connection to the network 110.

Optionally, the monitoring information is available for presentation by the monitoring device 101 having a user interface, for example, display. The display may be integrated in the monitoring device 101 and/or externally attached to the monitoring device 101.

Optionally, processing and/or analysis of the monitored respiratory sounds and/or the additional sensory data are performed by the client terminal 106 used by the caregiver 103. The sleep session information collected during 201 and 201A is transmitted from the monitoring device 101 to the client terminal 106 of the caregiver 103. The client terminal 106 executes an analysis application, such as the analysis module 121, which processes and/or analyzes the monitored data as described in 202 through 206 and presents the results to the caregiver 103.

Optionally, processing and/or analysis of the monitored respiratory sounds and/or the additional sensory data are performed at the central unit 104. The sleep session information collected during 201 and 201A is transmitted from the monitoring device 101 to the central unit 104 which executes an analysis application, such as the analysis module 122, which processes and/or analyzes the monitored data as described in 202 through 206 and presents the results to the caregiver 103. The information may be accessible by the caregivers 103 through a software application executed on the client terminal 106 and/or through web based service using a web browser executed on the client terminal 106.

According to some embodiments of the present invention, there are provided systems and methods for managing sleep sessions of patients 102 to enable one or more caregivers 103 to analyze, diagnose and/or be alerted of breathing disorder events of a plurality of patients. Managing the sleep session may present one or more sleep sessions of one or more patients 102 to the caregiver 103 using the one or more of the client terminals 106. The caregiver 103 may inspect any time period of the sleep session to diagnose the sleep breathing disorder and recommend a treatment. The caregiver 103 may adjust the time scale of the presentation so as to review a selected time period. The caregiver 103 may also review stimulation events which may have initiated following a sleep breathing disorder event. The sleep sessions, sleep breathing disorder events and/or stimulation events of the patients 102 may be prioritized for caregiver 103 to allow the caregiver 103 to respond to the plurality of events in an orderly manner. Prioritization may be done according to a plurality of conditions, for example, severity, history of sleep breathing disorder events and/or custom settings.

Notifications may be generated to indicate the caregiver 103 of a plurality of events, for example, sleep breathing disorder detection, sleep session start time, sleep session duration, respiratory sounds level and/or history information of the one or more patients 102. The notifications may be generated according to a plurality of notification rules which may be automatically set and/or adjusted by the one or more caregivers 103. The notification rules may have default values and/or may be adjusted automatically according to a plurality of parameters, for example, recent sleep breathing disorder events, frequency of sleep breathing disorder events and/or deterioration in the condition of the patient 102. Table 1 below provides an exemplary set of criteria and/or rules for initiating notifications to the one or more caregivers 103. Each parameter may be associated with a notification per a default value and/or a value adjusted by the one or more caregivers 103.

TABLE 1

| # | Criterion | Rule | Default | Notification |
|---|---|---|---|---|
| 10 | Session Begins | Ignore first <n> minutes | 15 | N/A |
| 11 | Session Ends | Ignore last <n> minutes | 5 | N/A |
| 20 | Unused | Patient did not use the monitoring | 7 | Yes, R3d, |
| | | application for <n> nights | | Ad |
| 30 | Ignore Short | Sleep Session is less <n> minutes | 120 | Yes, Aw |
| 31 | Short Session | Sleep session is less than <n> minutes | 240 | Yes, Aw |
| | | but more than {Ignore Short} | | |
| 32 | Long Session | Sleep session is more than <n> hours | 10 | No, |
| 33 | Many Shorts | In the last 7 sleep sessions, <n> sleep | 5 | Yes, Ad |
| | | sessions were {Short Session} | | |
| 34 | Many Longs | In the last 7 sleep sessions, <n> sleep | 5 | Yes, Ad |
| | | sessions were {Long Session} | | |
| 50 | Apnea | Average of over <n> suspected apnea | 20 | Yes, F30d |
| | | events per hour during a sleep session | | |
| 51 | Apnea Very | Increase of at least <n>% in {Apnea} | 40, 2, 7 | Yes, Ad |
| | Increase | in the last <s> sleep sessions compared | | |
| | | to average of previous <t> sleep | | |
| | | sessions | | |
| 52 | Apnea Increase | NOT {Apnea Very Increased} AND | 10, 2, 7 | Yes, Ad |
| | | increase of at least <n>% in apnea | | |
| | | events in the last <s> sleep sessions | | |
| | | compared to average of previous <t> | | |
| | | sleep sessions | | |
| 53 | Apnea Decrease | Decrease of at least <n>% in apnea | 10, 2, 7 | Yes, Ad |
| | | events in the last <s> sleep sessions | | |
| | | compared to average of previous <t> | | |
| | | sleep sessions | | |
| 70 | Noise | Increase of at least <n>dB in average | 15 | No, Aw |
| | | noise during the sleep session | | |
| 71 | Noise Increase | Increase of at least <n>% in noise in | 20, 2, 7 | No, Aw |
| | | the last <s> sleep sessions compared to | | |
| | | average of last <t> sleep sessions | | |
| 72 | Noise Decrease | Decrease of at least <n>% in noise in | 20, 2, 7 | No, Aw |
| | | the last <s> sleep sessions compared to | | |
| | | average of last <t> sleep sessions | | |
| 90 | Snoring Very | Increase of at least <n>% in snoring | 50, 2, 7 | Yes, Ad |
| | Increased | per hour in the last <s> sleep sessions | | |
| | | compared to average of last <t> sleep | | |
| | | session AND % of snoring per hour is | | |
| | | more than 10% | | |
| 91 | Snoring | NOT <Snoring Very Increased> AND | 20, 3, 7 | No, Aw |
| | Increased | an increase of at least <n>% in snoring | | |
| | | per hour in the last <s> sleep sessions | | |
| | | compared to average of last <t> sleep | | |
| | | session AND % of snoring per hour is | | |
| | | more than 10% | | |
| 92 | Snoring | Decrease of at least <n>% in snoring | 20, 3, 7 | No, Aw |
| | Decreased | in the last <s> sleep sessions compared | | |
| | | to average of previous <t> sleep | | |
| | | sessions | | |
| 110 | Wheezing Very | Increase of at least <n>% in wheezing | 50, 2, 7 | Yes, Ad |
| | Increased | per hour in the last <s> sleep sessions | | |
| | | compared to average of last <t> sleep | | |
| | | session AND % of wheezing per hour | | |
| | | is more than 10% | | |
| 111 | Wheezing | NOT <Wheezing Very Increased> | 20, 3, 7 | No, Aw |
| | Increased | AND an increase of at least <n>% in | | |
| | | wheezing per hour in the last <s> | | |
| | | sleep sessions compared to average of | | |
| | | last <t> session AND % of wheezing | | |
| | | per hour is more than 10% | | |
| 112 | Wheezing | Decrease of at least <n>% in wheezing | 20, 3, 7 | No, Aw |
| | Decreased | in the last <s> sleep sessions compared | | |
| | | to average of previous <t> sleep | | |
| | | sessions | | |
| 130 | Coughing Very | Increase of at least <n>% in coughing | 50, 2, 7, | Yes, Ad |
| | Increased | per hour in the last <s> sleep sessions | 100 | |
| | | compared to average of last <t> sleep | | |
| | | session AND # of coughing per hour is | | |
| | | more than <u> | | |
| 131 | Coughing | NOT <Wheezing Very Increased> | 20, 3, 7, | No, Aw |
| | Increased | AND an increase of at least <n>% in | 100 | |
| | | coughing per hour in the last <s> | | |
| | | sleep sessions compared to average of | | |
| | | last <t> sleep session AND # of | | |
| | | coughing per hour is more than <u> | | |
| 132 | Coughing | Decrease of at least <n>% in coughing | 20, 3, 7 | No, Aw |
| | Decreased | in the last <s> sleep sessions compared | | |
| | | to average of previous <t> sleep | | |
| | | sessions | | |
| 150 | Bruxism | Over <n> suspected episodes of | 20 | No, Ad |
| | | Bruxism during a sleep session | | |
| 151 | Bruxism | Increase of at least <n>% in | 30, 3, 7, | No, Ad |
| | Increased | {Bruxism} in the last <s> sleep | | |
| | | sessions compared to average of | | |
| | | previous <t> sleep sessions | | |
| 152 | Bruxism | Decrease of at least <n>% in Bruxism | 40, 3, 7 | No, Ad |
| | Decreased | in the last <s> sleep sessions compared | | |
| | | to average of previous <t> sleep | | |
| | | sessions | | |
| 170 | Talking | Over <n> suspected episodes of | 3 | No, Ad |
| | | talking out of sleep during a sleep | | |
| | | session | | |
| 171 | Talking | Increase of at least <n>% in talking out | 20, 3, 7, | No, Ad |
| | Increased | of sleep in the last <s> sleep sessions | | |
| | | compared to average of previous <t> | | |
| | | sleep sessions | | |
| 172 | Talking | Decrease of at least <n>% in talking | 40, 3, 7 | No, Ad |
| | Decreased | out of sleep in the last <s> sleep | | |
| | | sessions compared to average of | | |
| | | previous <t> sleep sessions | | |
| 190 | Compliance | Report if dental appliance has been | | No |
| | | used in a sleep session | | |
| 191 | Compliance | Report % of compliance on the last | 30 | No, Aw |
| | Period | <n> days (once after compliance data | | |
| | | is loaded) | | |

Notes:
1) The caregiver 103 may adjust rule parameters which are enclosed in < >, for example, <n>, <t>, <s> and/or <u>.
2) The default column provides exemplary default values for the rule associated with it (at the same row) in left to right order.
3) Notification notation index:
A denotes Aggregate. All notifications are aggregated into a single notification
Aw denotes Aggregate weekly. All notifications of the past week are aggregated into a single notification.
Ad denotes Aggregate daily. All notifications of the past day are aggregated into a single notification.
R denotes Repetitive. Notify repeatedly as long as the associated rule is true until notification is treated and/or removed. For example, R3d denote repeat every 3 days as long as rule is true.
F denotes Fresh. This is when the rule is true for the first time. For example, F30d denotes the associated rule is fulfilled for the first time or it turned true after being false for the past 30 days.

Optionally, alerts are generated to the caregiver 103 upon fulfillment of one or more alert conditions, for example, sleep breathing disorder detection, stimulation initiated and/or excessive levels of respiratory sounds are identified. The alerts may be generated in real time in case the respiratory patterns are available from the monitoring device 101 in real time and/or may be generated after the sleep session(s) are completed.

Optionally, history of the sleep sessions, including, for example, sleep session monitoring information, sleep breathing disorder events, stimulation events, previous diagnosis, previous treatment recommendations, previous treatment and/or follow up information is stored and made available to the caregiver 103.

As shown at 209, alerts may be generated to one or more caregivers 103 according to default alert conditions and/or according to alert conditions adjusted by the one or more caregivers 103.

As shown at 210, the monitoring information collected form one or more monitoring devices 101 is managed for the plurality of patients 102 for presentation and/or prioritization to allow the one or more caregivers 103 to diagnosis and/or recommend treatment for the one or more patients 102 by analyzing sleep sessions information. Notifications may be generated to the one or more caregivers 103 according to pre-defined notification rules, for example, sleep session duration, sleep breathing disorder events and/or excessive respiratory sounds level.

Optionally, further analysis is performed over the monitoring information, for example, statistics generation and/or more meticulous analysis. Statistics may provide for example, distribution of sleep breathing disorder events over multiple sleep session, distribution of sleep breathing disorder events over time during a single sleep session and/or distribution of sleep breathing disorder events per sleep breathing disorder type. The meticulous analysis may provide higher accuracy and/or validity of the suspected sleep breathing disorder events to allow the caregivers 103 to better diagnose and/or treat the patient 102.

Reference is now made to FIG. 2A which a flowchart of an exemplary process of monitoring and managing sleep breathing disorder events in which processing and analysis is performed at the caregiver client terminal, according to some embodiments of the present invention. According to some embodiments of the present inventions, processing and/or analysis of the monitoring information collected by the monitoring device 101 from the patient 102, is performed at the client terminal(s) 106 of the caregiver(s) 103. As shown at 201 through 201A, a process 200A for monitoring and managing sleep breathing disorders starts the same as the process 200 with collecting the respiratory sounds of the patient 102 and optionally collecting additional monitoring information, for example, chest movement, heart beat rate and/or blood oxygenation.

As shown at 201C, the monitoring device 101 transmits the monitoring information over a network such as the network 110 to one or more of the client terminals 106 of the caregivers 103.

As shown at 202 through 206, the process 200A continues the same as the process 200 with the analysis of the respiratory sounds and optionally the additional monitoring information performed at the client terminal 106.

As shown at 207, optionally, in case the monitoring device 101 transmits the monitoring information in real time to the client terminal 106, the client terminal 106 may, following detection of a suspected sleep breathing disorder event, direct the monitoring device 101 to initiate stimulation to the patient in order to cause the patient 102 to exit the sleep breathing disorder event.

As shown at 209, optionally, in case the monitoring device 101 transmits the monitoring information in real time to the client terminal 106, the client terminal 106 may, following detection of a suspected sleep breathing disorder event, generate an alert to one or more of the caregivers 103 to report the suspected sleep breathing disorder event.

As shown at 210, the monitoring information collected form one or more monitoring devices 101 is managed for the plurality of patients 102 for presentation and/or prioritization to allow the one or more caregivers 103 to diagnosis and/or recommend treatment for the one or more patients 102 by analyzing sleep sessions information. Notifications may be generated to the one or more caregivers 103 according to pre-defined notification rules, for example, sleep session duration, sleep breathing disorder events and/or excessive respiratory sounds level.

Reference is now made to FIG. 2B which a flowchart of an exemplary process of monitoring and managing sleep breathing disorder events in which processing and analysis is performed at the central unit, according to some embodiments of the present invention. According to some embodiments of the present inventions, processing and/or analysis of the monitoring information collected by the monitoring device 101 from the patient 102, is performed at the central unit 104. As shown at 201 through 201A, a process 200A for monitoring and managing sleep breathing disorders starts the same as the process 200 with collecting the respiratory sounds of the patient 102 and optionally collecting additional monitoring information, for example, chest movement, heart beat rate and/or blood oxygenation.

As shown at 201D, the monitoring device 101 transmits the monitoring information over a network such as the network 110 to the central unit 104.

As shown at 202 through 206, the process 200B continues the same as the process 200 with the analysis of the respiratory sounds and optionally the additional monitoring information performed at the central unit 104.

As shown at 207, optionally, in case the monitoring device 101 transmits the monitoring information in real time to the central unit 104, the client terminal 104 may, following detection of a suspected sleep breathing disorder event, direct the monitoring device 101 to initiate stimulation to the patient in order to cause the patient 102 to exit the sleep breathing disorder event.

As shown at 209, optionally, in case the monitoring device 101 transmits the monitoring information in real time to the central unit 104, the central unit 104 may, following detection of a suspected sleep breathing disorder event, generate an alert to one or more of the caregivers 103 to report the suspected sleep breathing disorder event.

As shown at 210, the monitoring information collected form one or more monitoring devices 101 is managed for the plurality of patients 102 for presentation and/or prioritization to allow the one or more caregivers 103 to diagnosis and/or recommend treatment for the one or more patients 102 by analyzing sleep sessions information. Notifications may be generated to the one or more caregivers 103 according to pre-defined notification rules, for example, sleep session duration, sleep breathing disorder events and/or excessive respiratory sounds level.

Optionally, the analysis of the respiratory sounds and/or the additional monitoring information, the alert generation and/or the management of the plurality of patients is distributed between the monitoring device 101, one or more of the client terminals 106 and/or the central unit 104. The above mentioned operations may be associated with software application programs which may execute on one or more of the processing units included in the monitoring device 101, one or more of the client terminals 106 and/or the central unit 104.

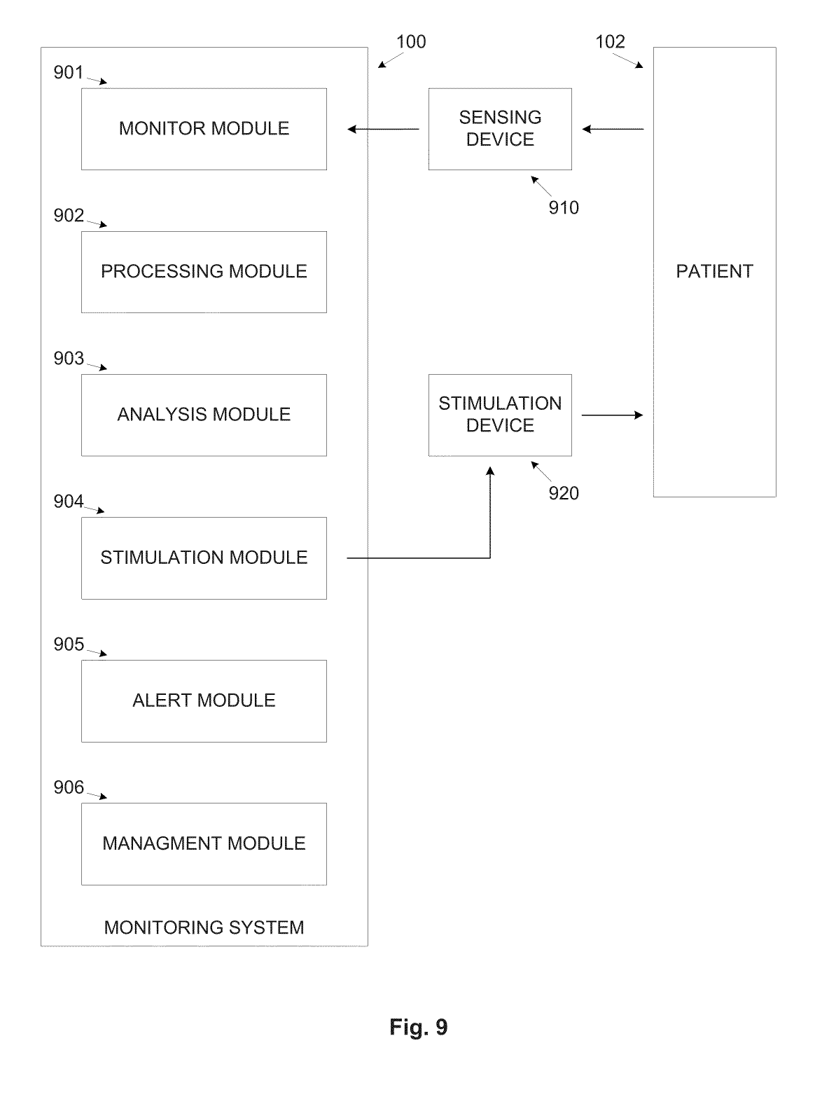

Reference is now made to FIG. 9 which is a schematic illustration of exemplary modules for implementing an exemplary system for monitoring and managing sleep breathing disorders, according to some embodiments of the present invention. The system 100 for monitoring and managing sleep breathing disorders may include one or more software modules for performing the monitoring, analysis and/or management of sleep breathing disorders. The software modules may constitute all or part of the analysis modules 120, 121 and/or 122. A monitor module 901 which executes on the monitoring device 101 collects monitoring information from one or more sensors 910 that monitor the patient 102. The monitoring information may be transmitted from the monitoring device 101 to one or more of the client terminals 106 and/or the central unit 104. The collected monitoring information is processed by a processing module 902 which executes on the monitoring device 101 to extract a pattern of the one or more vital life signs, for example, respiratory sounds, heart beat rate, oxygenation level in the blood and/or chest movement. An analysis module 903 which executes on the monitoring device 101 analyses the one or more patterns to identify one or more sleep breathing disorder events. A management module 906 manages the information relating to sleep breathing disorders of the one or more patients 102, for example, collected monitoring information, history records of sleep breathing disorders, previous diagnosis and/or previous treatment. the management module 906 may initiate notifications to the one or more caregivers 103 to indicate of a plurality of events relating to sleep breathing disorders of the one or more patients 102, for example, sleep session duration, sleep breathing disorder events and/or excessive respiratory sounds level.

Optionally, a stimulation module 904 executes on the monitoring device 101. The stimulation module 904 may initiate stimulation to the patient 102 during a sleep breathing disorder event in order to cause the patient 102 to exit the sleep breathing disorder event. The stimulation module 904 initiates the stimulation through a stimulation device 1020 integrated in the monitoring device 101 and/or communicating and/or connected to the monitoring device 101.

Optionally, an alert module 905 executes on the monitoring device 101. The alert module 905 may generate alerts to the one or more caregivers 103 according to the plurality of alert rules which are automatically set and/or adjusted by the one or more caregivers 103.

The system 100 may be a distributed system in which one or more of the software modules are executed on one or more of the processing units of the system 100. The processing module 902, the analysis module 903, the alert module 905 and/or the management module 906 may be software applications that execute on the one or more client terminals 106 used by the one or more caregivers 103. The processing module 902, the analysis module 903, the alert module 905 and/or the management module 106 may have access to the monitoring device 101 the network 110. The management module 906 may have access to the data repository 105 to allow the one or more caregivers 103 to review history records of the one or more patients 102. The monitoring information and/or the analysis of the monitoring information may be presented by the client terminal 106 to the caregivers 103.

Optionally, the processing module 902, the analysis module 903, the alert module 905 and/or the management module 906 are executed on the central unit 104 and are accessible to the one or more caregivers 103 as a web based service through the client terminal 106 using, for example, a web browser.

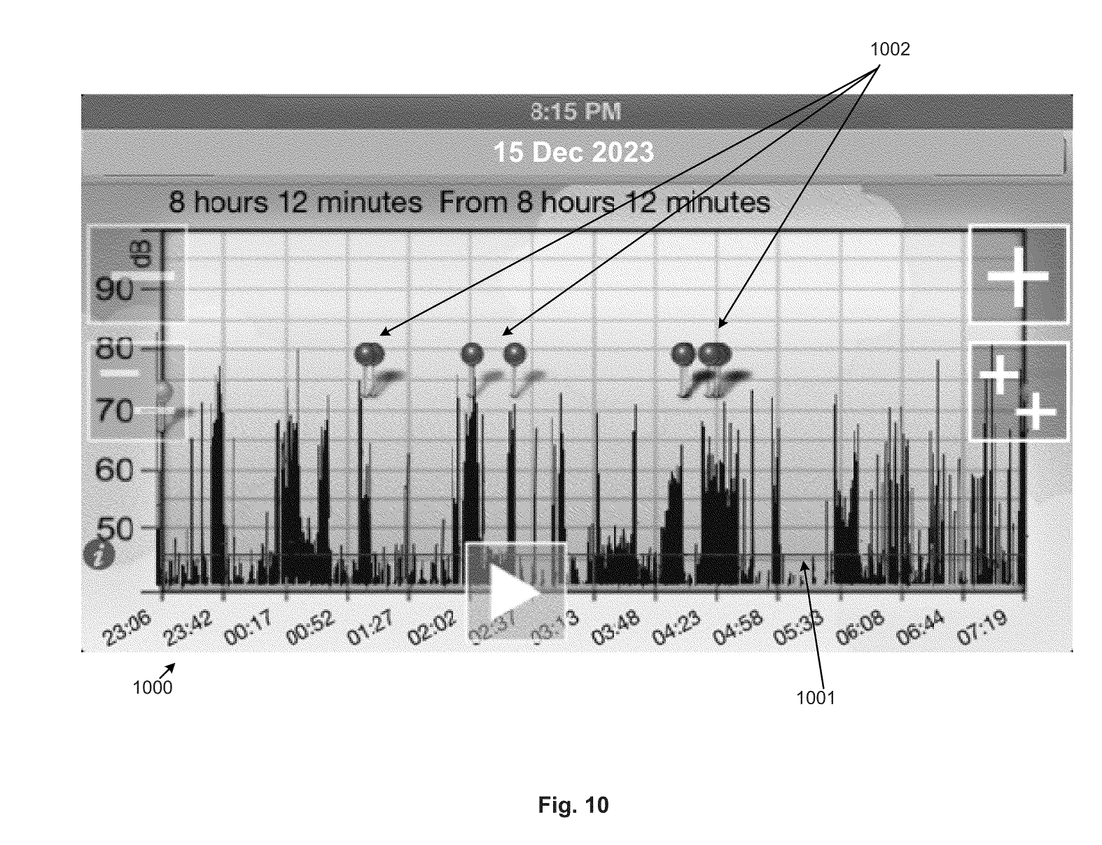

Reference is now made to FIG. 10 which is a screen capture of a monitored exemplary sleep session as presented by an exemplary user interface of an exemplary monitoring device, according to some embodiment of the present invention. A screen capture 1000 presents respiratory sounds level during an exemplary sleep session as displayed by an exemplary user interface on the monitoring device 101. A dynamic threshold level 1001 is maintained at 46 for most of the session. A plurality of pins 1002 indicates a plurality of stimulation events, each following a sleep breathing disorder event.

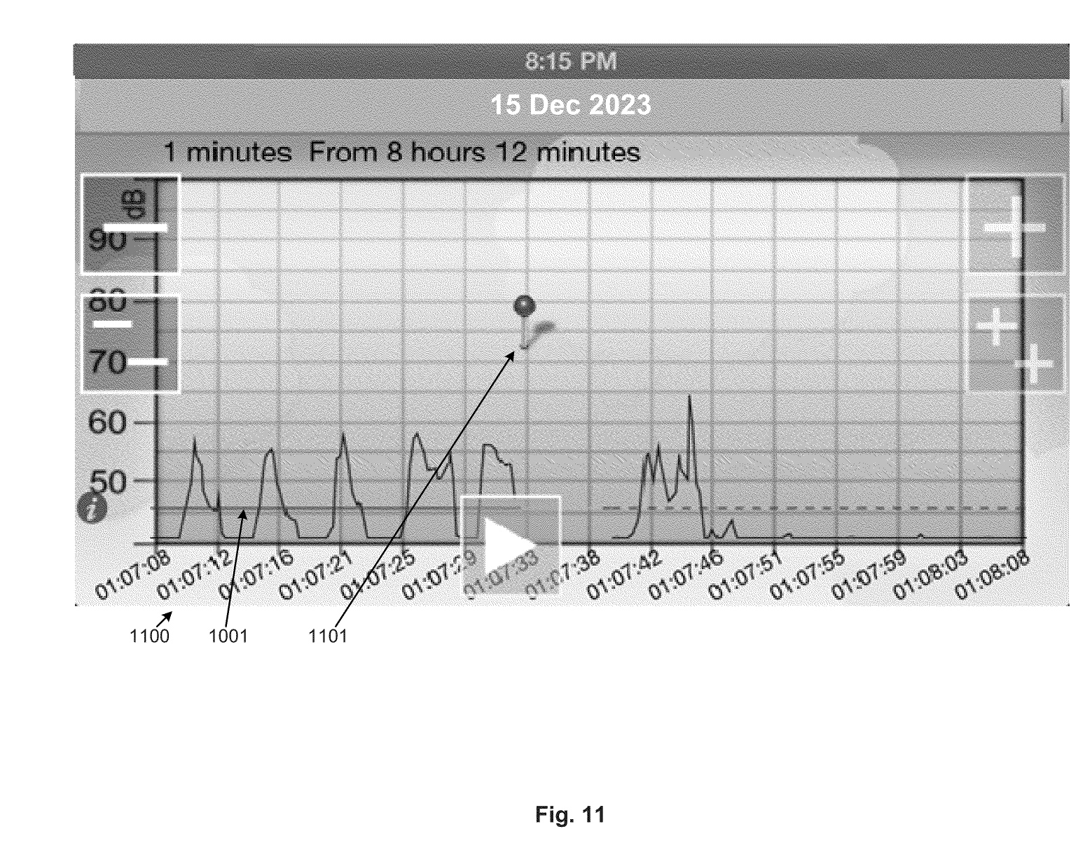

Reference is now made to FIG. 11 which is a screen capture of a result of an exemplary stimulation during a sleep session as presented by an exemplary user interface of an exemplary monitoring device, according to some embodiment of the present invention. A screen capture 1100 presents a zoom in view of respiratory sounds during a partial sleep session in which a stimulation event 1101 causes the patient 102 to exit the sleep breathing disorder event. As is shown in the screen capture 1100, prior to the stimulation event 1101 the respiratory sounds exceed the dynamic threshold 1001. After the stimulation event 1101 there is a momentary noise which may indicate movement of the patient 102, followed respiratory sounds lower than the dynamic threshold 1001.

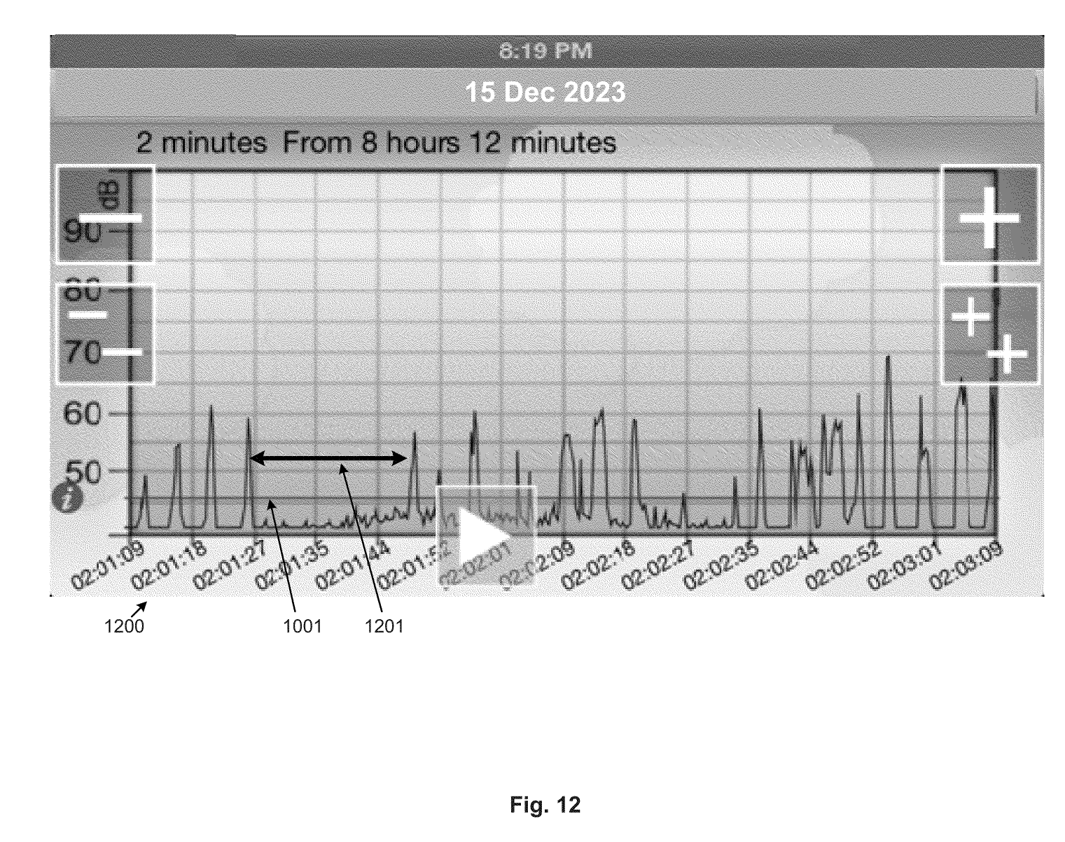

Reference is now made to FIG. 12 which is a screen capture of an exemplary candidate for an apnea event as presented by an exemplary user interface of an exemplary monitoring device, according to some embodiment of the present invention. A screen capture 1200 presents respiratory sounds during a 2 minutes time period out of a sleep session in which a candidate for an apnea 1201 event is identified.

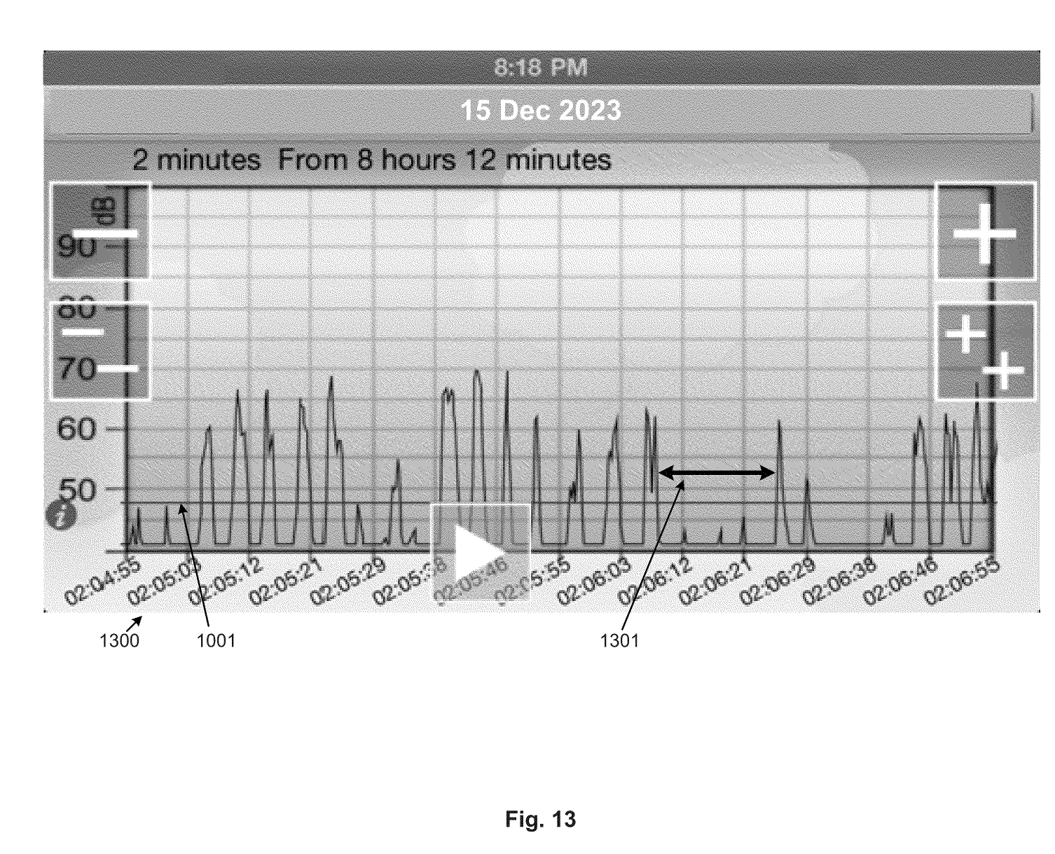

Reference is now made to FIG. 13 which is a screen capture of an exemplary candidate for a hypopnea event as presented by an exemplary user interface of an exemplary monitoring device, according to some embodiment of the present invention. A screen capture 1300 presents respiratory sounds during a 2 minutes time period out of a sleep session in which a candidate for a hypopnea 1301 event is identified.

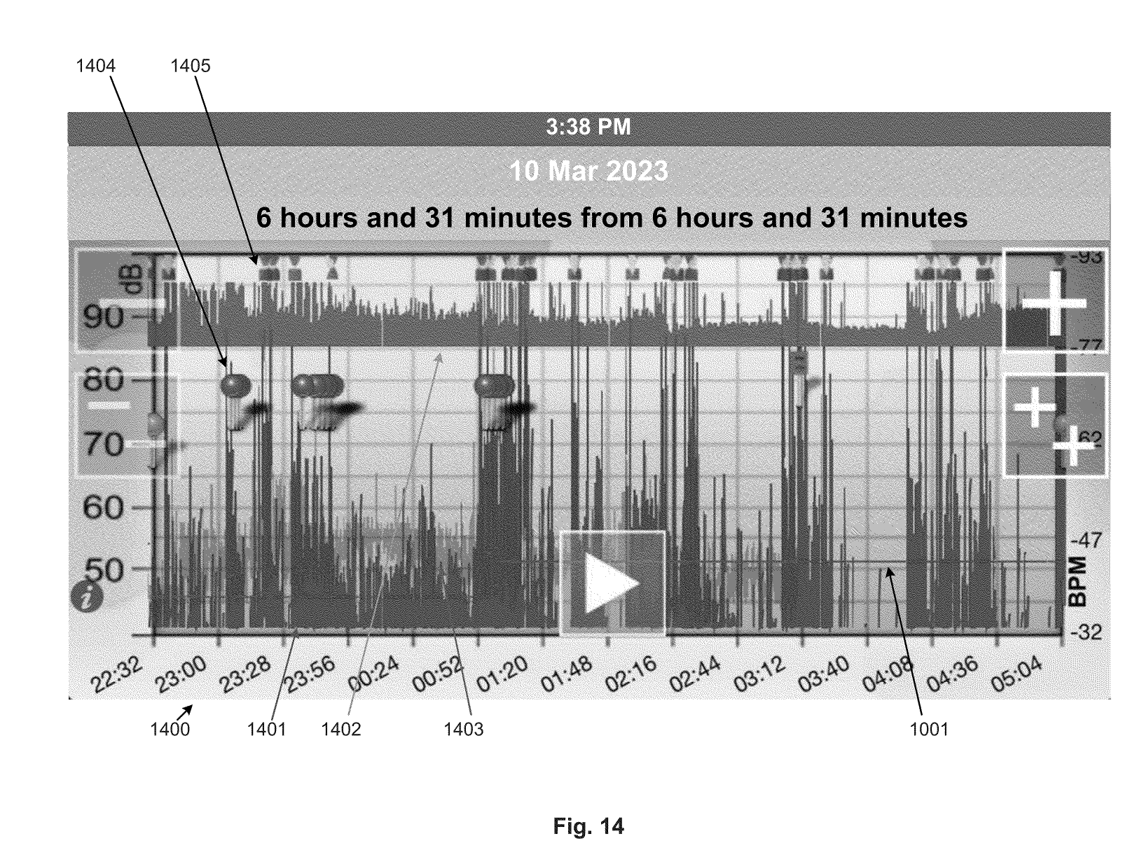

Reference is now made to FIG. 14 which is a screen capture of a monitored exemplary sleep session with multiple sensors as presented by an exemplary user interface of an exemplary monitoring device, according to some embodiment of the present invention. A screen capture 1400 presents a sleep session in which respiratory sounds, heart beat rate and chest movements are monitored. The screen capture presents a respiratory sound level graph 1401 (blue), a chest movement graph 1402 (green), a heartbeat rate graph 1403 (red), a plurality of pins, such as 1404 indicating stimulation events and posture symbols, such as 1405 indicating the posture of the patient 102. A scale for the heartbeat rate (beat per minute—BPM) is provided on the right hand side of the screen capture 1400. It may be seen in the screen capture 1400 that when a sleep breathing disorder event is identified, stimulation 1404 may be generated to the patient 102 in order to cause the patient 102 to exit the sleep breathing disorder situation. The posture indications 1405 may enhance the analysis of the chest movement input and may also indicate the effect of the stimulation as the patient 102 may switch his posture as result of the stimulation and may exit the sleep breathing disorder situation.

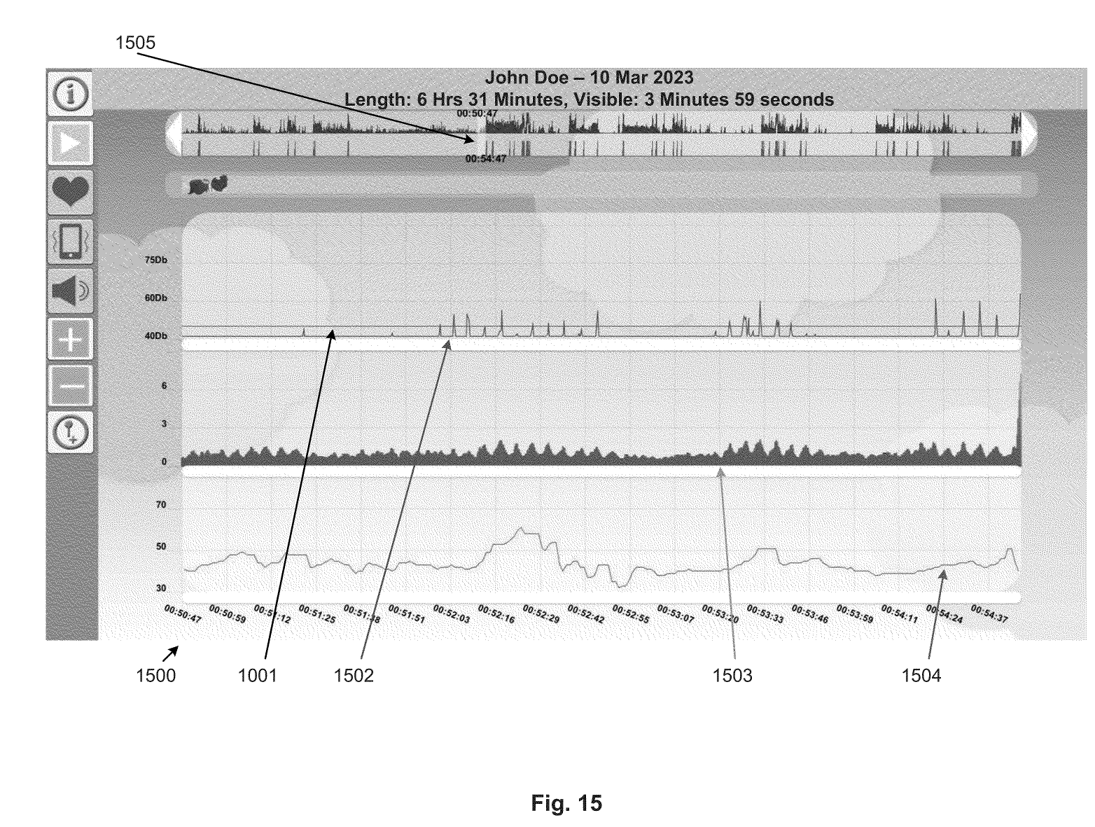

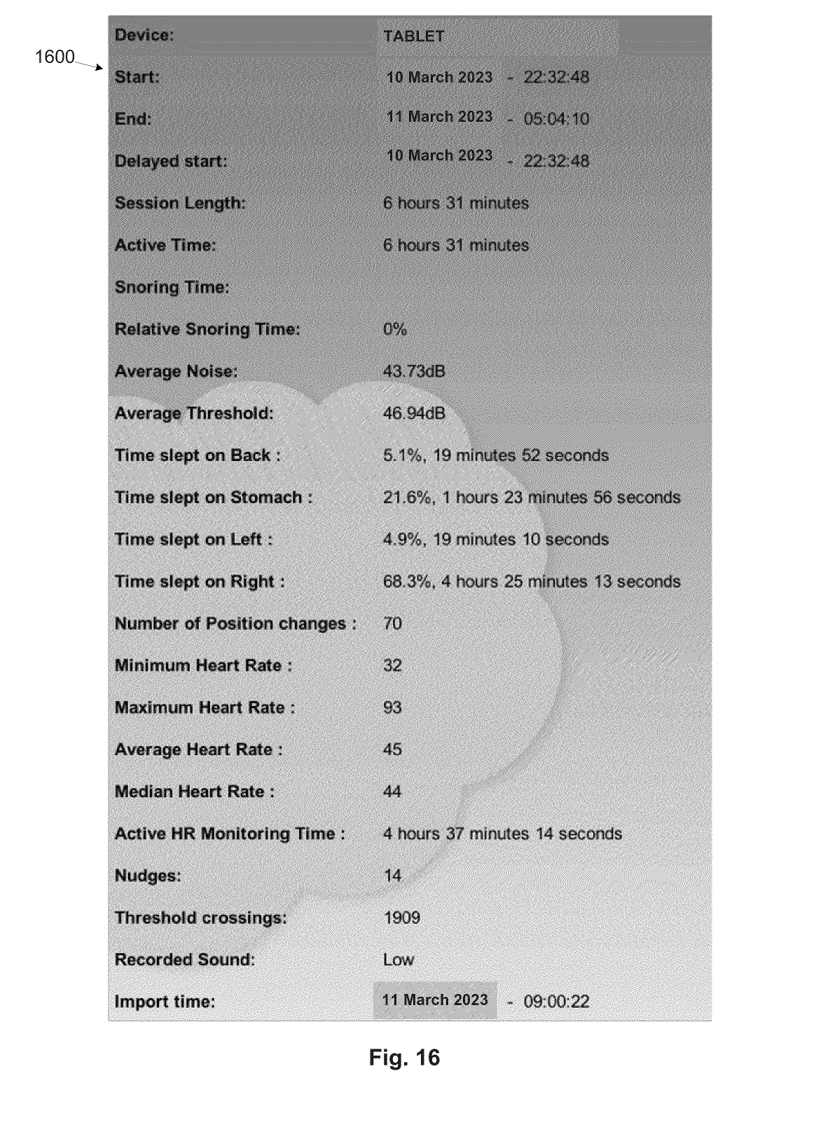

Reference is now made to FIG. 15 which is a screen capture of a monitored exemplary sleep session as presented by an exemplary user interface of an exemplary client terminal 106 used by a caregiver, according to some embodiment of the present invention. A screen capture 1500 presented by an exemplary user interface on the client terminal 106 of the caregiver 103 provides the same information as presented, for example, by the screen capture 1400. The screen capture 1500 presents the respiratory sound levels through a graph 1502, the dynamic threshold through a line 1001, chest movement indications as depicted through a graph 1503 and heartbeat rate through a graph 1504. An appropriate scale is provided for each of the measured indications. As shown at a top bar of the screen capture 1500, a user friendly zoom-in/zoom-out tool is available for the caregiver 103 to adjust the view of the monitored sleep session. An indication 1505 identifies the specific time interval out of the sleep session that is presented in detail on the screen.

Reference is now made FIG. 16 which is a screen capture of an exemplary statistics summary of a monitored exemplary sleep session consisting as presented by an exemplary user interface of an exemplary monitoring device, according to some embodiment of the present invention. A screen capture 1600 provides a statistics summary of an exemplary sleep session. The screen capture 1600 provides a plurality of identification details of the exemplary sleep session, for example, monitoring device type and/or model, start and/or end time of the sleep session, duration of the sleep session and/or the reception time of the report of the monitored sleep session. The screen capture 1600 provides a plurality of statistics information items extracted from the monitoring data of the sleep session, for example, average noise, average level of the dynamic threshold, duration of sleep at specific postures, number of posture switch, average heartbeat rate and/or number of stimulation events (nudges).

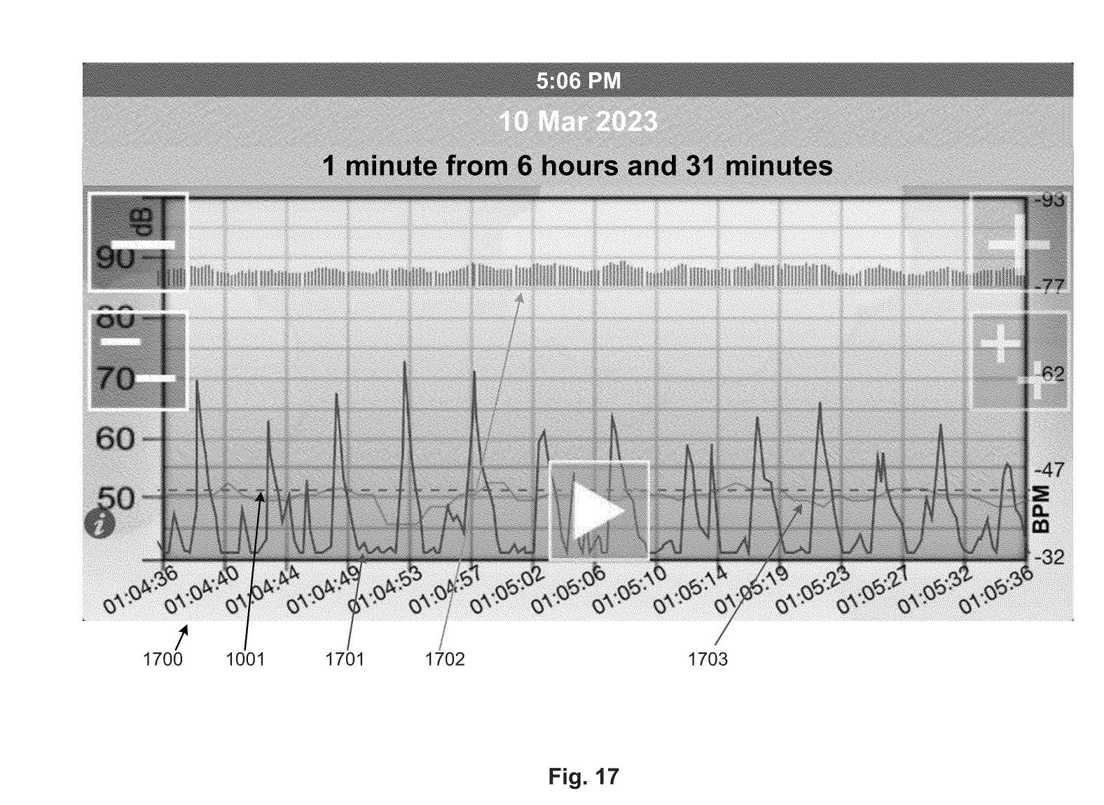

Reference is now made to FIG. 17 which is a screen capture of a zoom in view of a monitored exemplary sleep session as presented by an exemplary user interface of an exemplary monitoring device, according to some embodiment of the present invention. A screen capture 1700 presents a zoom-in view of a one minute period of an exemplary sleep session which includes snoring sounds. The screen capture 1700 presents respiratory sounds level through a graph 1701, the dynamic threshold through the line 1001, chest movements through a graph 1702 and heartbeat rate through a graph 1703. As seen in the screen capture 1700, the graph 1701 exceeds the dynamic threshold 1001 indicating excessive respiratory sounds level, such as snoring. Chest movements as indicated by the graph 1702 are rapid further indicating the snoring as is evident from the recorded respiratory sounds level. However the heartbeat rate as indicated through the graph 1703 shows very little variation during the sleep session period.

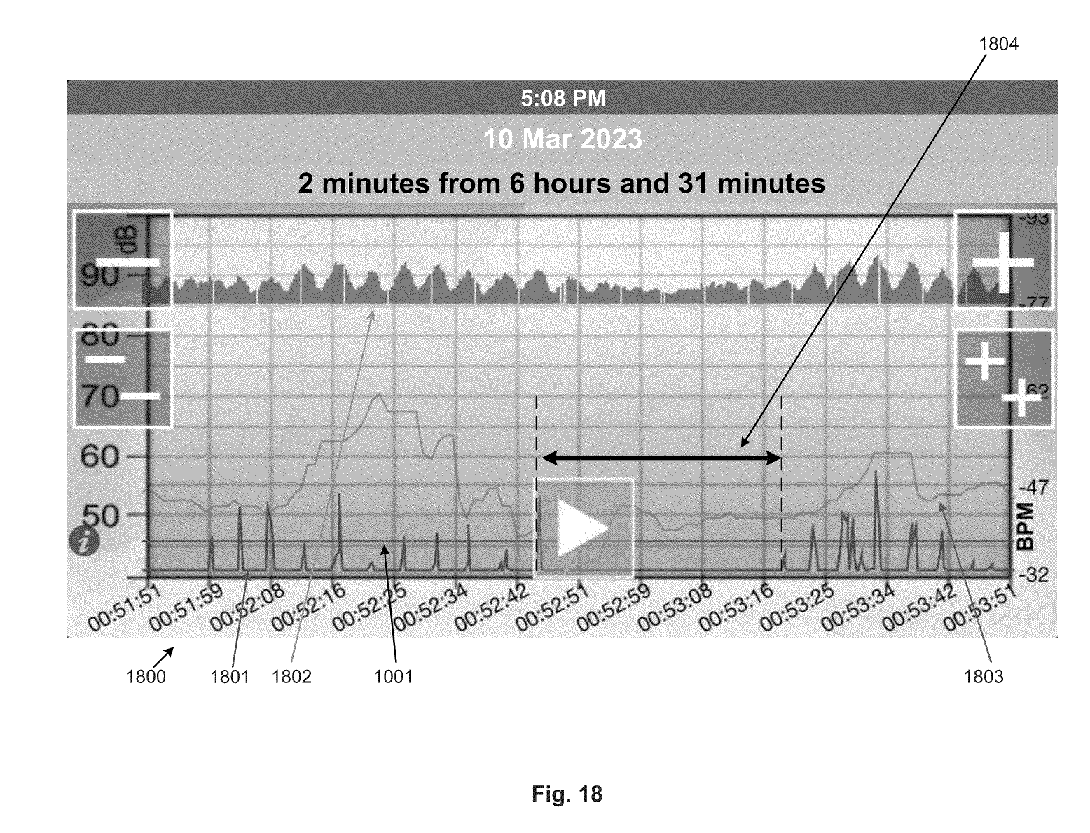

Reference is now made to FIG. 18 which is a screen capture of a suspected apnea event identified during an exemplary sleep session using multiple sensors as presented by an exemplary user interface of an exemplary monitoring device, according to some embodiment of the present invention. The screen capture 1800 presents zoom-in view of a two minutes period of an exemplary sleep session in which multiple sensors indications are available. As seen in the screen capture 1800, respiratory sounds level, for example, snoring as described by a graph 1801 exceeds the dynamic threshold 1001, while chest movement shown through a graph 1802 indicate movement which falls in line with the snoring. The heartbeat rate as described by a heartbeat graph 1803 shows increasing heartbeat rate over time. As shown at 1804 respiratory sounds level drops below the dynamic threshold 1001 indicating a suspected apnea sleep breathing disorder event. Chest movement and heartbeat rate during the time period 1804 are also reduced, which may indicate that respiratory is not performed by the monitored patient 102. Following the time period 1804 respiratory sounds level is increased and is accompanied with increased chest movement and heartbeat rate suggesting the patient 102 may resumed respiratory action.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant systems, methods and computer programs will be developed and the scope of the term commerce information and price is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of monitoring a patient to identify a sleep breathing disorder, comprising:
collecting, using a handheld monitoring device, a plurality of respiratory sounds of a patient during a sleep session using at least one sensor, said at least one sensor connects to said handheld monitoring device;
recording environment noise during said sleep session and averaging said environment noise to set an average noise level;
calculating a dynamic threshold according to said average noise level;
deriving a plurality of amplitudes from said plurality of respiratory sounds according to said dynamic threshold; and
identifying a sleep breathing disorder event by analyzing a pattern of said plurality of amplitudes.

2. The method of claim 1, wherein identifying said at least one sleep breathing disorder event includes avoiding a false detection of said at least one sleep breathing disorder event by analyzing said pattern.

3. The method of claim 1, further comprising transmitting said pattern to at least one caregiver, said at least one caregiver diagnoses said at least one sleep breathing disorder event by analyzing said pattern.

4. The method of claim 3, further comprising generating at least one alert to said at least one caregiver when said at least one sleep breathing disorder event occurs.

5. The method of claim 1, further comprising initiating stimulation to said patient to cause said patient to exit said at least one sleep breathing disorder event by exciting said patient through a physical intervention.

6. The method of claim 1, wherein said sensor includes a microphone sampling said respiratory sound.

7. The method of claim 6, wherein said plurality of amplitudes is corrected to reduce fading effects of said microphone by analyzing timing characteristics of previous amplitudes of said plurality of amplitudes.

8. The method of claim 1, wherein said plurality of respiratory sounds include at least one member of a group consisting of: inhalation sound, exhalation sound, respiratory pause, snore sound and wheeze sound.

9. The method of claim 1, further comprising collecting a plurality of chest movement measurements to identify excessive chest movement which is indicative of said at least one sleep breathing disorder event by analyzing said plurality of chest movement measurements.

10. The method of claim 1, further comprising collecting a plurality of heart beat rate measurements to identify increased heart beat rate which is indicative of said at least one sleep breathing disorder event by analyzing said plurality of heart beat rate measurements.

11. The method of claim 1, further comprising collecting a plurality of blood oxygenation level measurements to identify decreased blood oxygenation level which is indicative of said at least one sleep breathing disorder event by analyzing said plurality of blood oxygenation level measurements.

12. The method of claim 1, wherein said handheld monitoring device executes at least one software application program for collecting said plurality of respiratory sounds, recording said environment noise, calculating said dynamic threshold, deriving said plurality of amplitudes, identifying said at least one sleep breathing disorder and transmitting said pattern to at least one caregiver.

13. A method of prioritizing diagnosis of sleep breathing disorder events of a plurality of patients, comprising:
collecting a plurality of sleep session records from a plurality of patients using a plurality of client terminals;
identifying a plurality of sleep breathing disorder events by analyzing said plurality of sleep session records; and
prioritizing said sleep breathing disorder events in a priority order according to severity of said plurality of sleep breathing disorder events; and
notifying at least one caregiver of said plurality of sleep breathing disorder events in said priority order by transmitting at least one alert to said at least one caregiver.

14. The method of claim 13, wherein said at least one caregiver diagnoses said plurality of sleep breathing disorder by analyzing said plurality of sleep session records.

15. The method of claim 13, wherein said at least one caregiver adjusts a plurality of alert rules to define at least one condition for transmitting said at least one alert.

16. The method of claim 15, wherein said plurality of alert rules are set automatically to default values.

17. The method of claim 13, wherein said priority order is set automatically according to sleep breathing disorder history of said plurality of patients.

18. A system for monitoring sleep breathing disorders of patients, comprising:
a monitoring module which collects a plurality of respiratory sounds of at least one patient using at least one sensor;
a processing module which identifies a plurality of amplitudes derived from said plurality of respiratory sounds by dynamically adjusting a threshold to reduce environment noise;
an analysis module which identifies at least one sleep breathing disorder event by analyzing a pattern of said plurality of amplitudes;
a management module which aggregates said pattern for presentation to at least one caregiver for diagnosis; and
an alert module which informs said at least one caregiver of said at least one sleep breathing disorder by generating at least one alert to said at least one caregiver.

19. The system of claim 18, wherein said management module includes a user interface module for presenting said presentation to said at least one caregiver.

20. The system of claim 18, wherein said at least one caregiver adjusts at least one alert condition by adjusting a plurality of alert rules through said management module.

21. The system of claim 20, wherein said management module automatically sets said plurality of alert rules according to sleep breathing disorder history of said at least one patient, said sleep breathing disorder history is stored by said management module.

22. The system of claim 18, further comprising a stimulation module which initiates a stimulation to said at least one patient to cause said at least one patient to exit said at least one sleep breathing disorder event, said stimulation is generated through at least one stimulation device.

23. The system of claim 18, wherein said system is a distributed system comprising of at least one handheld device which identifies said at least one sleep breathing disorder event by monitoring said at least one patient and at least one client terminal used by said at least one caregiver for managing said at least one sleep breathing disorder event, said at least one handheld device and said at least one client terminal communicate with each other using at least one of a plurality of networks.

24. The system of claim 23, wherein said distributed system includes a central unit which provides management services, said central unit communicates with said at least one handheld device and said at least one client terminal using at least one of said plurality of networks.
